(19) United States
(12) Patent Application Publication
(10) Pub. No.: US 2011/0137177 A1
(43) Pub. Date: Jun. 9, 2011

(54) OPTICAL-COMBINED IMAGING METHOD, OPTICAL-COMBINED IMAGING APPARATUS, PROGRAM, AND INTEGRATED CIRCUIT

(76) Inventors: Tadamasa Toma, Osaka (JP); Satoshi Kondo, Kyoto (JP); Jun Cheng, Singapore (SG); Zhongyang Huang, Singapore (SG); Sheng Mei Shen, Singapore (SG)

(21) Appl. No.: 13/056,820

(22) PCT Filed: Jun. 9, 2010
(86) PCT No.: PCT/JP2010/003828

§ 371 Date: Jan. 31, 2011

(30) Foreign Application Priority Data
Jun. 10, 2009 (JP) 2009-139675

(51) Int. Cl. A61B 6/00

(52) U.S. Cl. 600/473

(57) ABSTRACT

An optical-combined imaging method which improves image quality and shortens the time for imaging includes: a structure identification step (S21) of identifying the position and size of an observation target object which is located within body tissue; a region determining step (S22) of determining an imaging subject region which includes the observation target object, based on the location and size identified in the structure identification step (S21); a measurement step (S23) of measuring diffused light propagated through the imaging subject region determined in the region determining step (S22); an imaging step (S24) of estimating an optical characteristic within the imaging subject region based on the measuring result in the measurement step (S23) and imaging the optical characteristic; and a displaying step (S25) of displaying the optical characteristic imaged in the imaging step (S24).

### TECHNICAL FIELD

The present invention relates to an optical-combined imaging method and optical-combined imaging apparatus for imaging optical characteristics within a living body a using diffused light.

### BACKGROUND ART

As in vivo imaging methods, X-rays, ultrasound, and the like, have seen widespread use centering on medical applications. Recently, however, near infrared light (NIR) having a wavelength of approximately 700 to 900 nm has been gathering attention as a noninvasive imaging technique. Near infrared light is suited to in vivo imaging because of relatively high in vivo permeability due to low absorption by water, and the like, and also because energy is low. Therefore, the distribution of the absorption coefficients or scattering coefficients in body tissues can be obtained by emitting near infrared light from the surface of a living body, and detecting the light which has been diffused and absorbed within the living body and which has once again returned to the surface of the living body. Such an imaging method is called diffuse optical tomography (DOT) due to the obtainment of three-dimensional information of the inside of the living body, using diffused light.

For example, since new blood vessels are formed at a malignant tumor area and the surroundings thereof following active proliferation of cancer cells, blood flow increases and hemoglobin concentration rises compared to that in normal tissues or benign tumors. Since the optical absorption coefficient for near infrared light increases when the hemoglobin concentration rises, detection of malignant tumors or judgment between a benign tumor and a malignant tumor becomes possible by checking the absorption coefficient distribution within the living body. In this manner, diffuse optical tomography allows for the obtainment of optical characteristics of body tissue that are unobtainable through x-ray or ultrasound, and thus offers much promise for applications to early cancer discovery, non-invasive biopsy, or monitoring chemotherapy results, and so on. At present, research centered on breast cancer in particular is actively taking place.

FIG. 1 is a schematic diagram showing a configuration example for diffuse optical tomography. First, a probe including optical input channels and optical output channels is held against the body surface of a test subject. Laser light, as input light, is emitted into the body of the test subject from the optical input channels which are connected to a light source, the light which is diffused by the body tissue reaches the tumor area. Furthermore, part of the light that has reached the tumor area reaches the optical output channels as output light, while being scattered and absorbed again. An image reconstructing device images (reconstructs) the distribution of the absorption coefficients or scattering coefficients within the living body based on information such as the attenuation level of the amplitude or the amount of change in phase of output light measured at the optical output channels with respect to input light emitted from the optical input channels.

The following is a general description of an example of an image reconstruction method in diffuse optical tomography. The unknowns are the distributions of the absorption coefficients and scattering coefficients within the body tissue, and at the time of image reconstruction, a three-dimensional imaging subject region is segmented into minute regions (hereinafter called voxels) and the absorption coefficient and scattering coefficient of each voxel is estimated. Although several models have been proposed for the stage of light propagation within the living body, a method called diffusion approximation is common as an accurate approximation method. Here, when an estimate value is given as the absorption coefficient and the scattering coefficient of each voxel, the light propagation region with the living body and the amplitude attenuation can be determined through diffusion approximation, and thus it is possible to estimate the amount of change in the phase and amplitude in the output light measured at the optical output channels with respect to the input light emitted from the optical input channels. Therefore, by iteratively updating the absorption coefficient and scattering coefficient of each voxel so that the estimate value for the amount of change in the phase and amplitude approaches or matches the actual measured value, the final estimate values of the absorption coefficient and scattering coefficient can be obtained. Here, the measurement of the amount of change in the phase and amplitude and model-based estimate value calculation are performed for pairs of the optical input channels and optical output channels.

In this manner, since image reconstruction in diffuse optical tomography is equivalent to solving an inverse problem, appropriately setting the initial value in iterative computation holds much promise for the improvement of reconstruction accuracy (estimation accuracy), in other words, the improvement of detection sensitivity and spatial resolution, and for the reduction in the amount of computational processing following the reduction in the number of iterations.

As an example of prior art, there is a method described in Patent Literature 1 which combines the use of ultrasound and diffuse optical tomography, and sets the initial value of the iterative computation based on a tumor location obtained through ultrasound. A method (optical-combined imaging method) which combines the use of a non-optical imaging method such as ultrasound and diffuse optical tomography is considered to be a very promising imaging method in the future in terms of being able to improve the performance of diffuse optical tomography itself, aside from providing functional information such as optical characteristics in addition to information, such as tissue structural information, provided by conventional diagnostic apparatuses such as ultrasound.

Here, the optical-combined imaging apparatus and the method thereof in aforementioned Patent Literature 1, which combines the use of a non-optical imaging method such as ultrasound and diffuse optical tomography shall be described.

FIG. 2 is a block diagram showing a configuration example of an optical-combined imaging apparatus 1000 in the aforementioned Patent Literature 1. An ultrasound signal measurement unit 1001 and an ultrasound signal processing unit 1002 are processing units for processing ultrasound signals. The ultrasound signal processing unit 1002 analyzes and images an ultrasound signal measured by the ultrasound signal measurement unit 1001, outputs ultrasound image data 1021 to a display unit 1003, and outputs, to an image reconstruction unit 1005, tumor information 1022 which indicates the location and size of a tumor. An optical signal measurement unit 1004 and the image reconstruction unit 1005 correspond to diffuse optical tomography processing units. The image reconstruction unit 1005 reconstructs the absorption coefficients within the living body, that is, estimates the absorption coefficients and outputs, to the display unit 1003, optical image data 1051 which represents the result of the estimation as an image. The display unit 1003 displays the ultrasound image data 1021 and the optical image data 1051.

FIG. 3 is flowchart showing operations in optical diffusion tomography in an optical-combined imaging method in the aforementioned Patent Literature 1. First, in step S1001, optical signals (diffused light) are measured in all of the channel pairs (pairs of the optical input channels and optical output channels). Next, in step S1002, the absorption coefficients of voxels within a predetermined region are calculated, that is, the absorption coefficients are reconstructed, using the measurement results from step S1001. Although the absorption coefficients are calculated by iterative computation, the initial value thereof is determined based on the tumor information 1022. Lastly, in step S1003, the absorption coefficient of each voxel is imaged and displayed.

FIG. 4A is a diagram showing an example of an external appearance of a combined probe that combines respective optical and ultrasound probes. The optical input channels are located on the left-side and the optical output channels are located at the right side, with the ultrasound probe sandwiched in between.

FIG. 4B is a diagram showing an example of light propagation regions between optical input channels and optical output channels. Each of the light propagation regions is created, for example, between a channel pair of an optical input channel In1 and an optical output channel Ou1, a channel pair of an optical input channel In2 and an optical output channel Ou2, and a channel pair of an optical input channel In3 and an optical output channel Ou3 in FIG. 4A. Generally, light emitted from an optical input channel draws a banana-shaped arc within the living body and reaches an optical output channel. Therefore, light is propagated through a deeper region as the distance between the optical input channel and the optical output channel in a channel pair increases. For example, light is propagated through a deep region 1 between the optical input channel In1 and the optical output channel Ou1, and is propagated through a shallow region 2 between the optical input channel In2 and the optical output channel Ou2, and propagated to a shallower region 3 between the optical input channel In3 and the optical output channel Ou3.

### CITATION LIST

### Patent Literature

[PTL 1] United States Patent Application Publication No. 2004/0215072

### Non Patent Literature

[NPL 1] IEEE SIGNAL PROCESSING MAGAZINE, NOVEMBER 2001, pp. 57-75

### SUMMARY OF INVENTION

### Technical Problem

However, the optical-combined imaging apparatus 1000 in the aforementioned Patent Literature 1 has the problem that the image quality of images generated by imaging deteriorates and a long time is required for imaging. This is because the diffuse optical tomography of the optical-combined imaging apparatus 1000 always uses all the channel pairs, and thus reconstructs the absorption coefficients for a fixed imaging subject region. Hereinafter, the problem of the optical-combined imaging apparatus 1000 in the aforementioned Patent Literature 1 shall be discussed specifically.

FIG. 5A is a diagram showing the positional relationship of a tumor with respect to an imaging subject region. As shown in FIG. 5A, with the optical-combined imaging apparatus 1000 in the aforementioned Patent Literature 1, the imaging subject region is fixed regardless of the location or size of the tumor.

FIG. 5B is a diagram showing the positional relationship between a tumor and light propagation regions in an imaging subject region. It should be noted that FIG. 5B shows an example of the light propagation regions of channel pairs in an xz direction cross-section of the imaging subject region shown in FIG. 5A.

As described above, light is propagated through a deep region 1 between the optical input channel In1 and the optical output channel Ou1, and is propagated through a shallow region 2 between the optical input channel In2 and the optical output channel Ou2, and propagated to a shallower region 3 between the optical input channel In3 and the optical output channel Ou3. It should be noted that although, in actuality, there are also regions through which light is propagated between other pairings (channel pairs) such as the pairing of the optical input channel In1 and the optical output channel Ou2, these are not illustrated in FIG. 5B in order to simplify description.

When region 3 and region 1 are compared, region 1 has a longer light propagation distance and thus light attenuation due to light absorption and scattering is greater, and as a result, light intensity detected or measured at the optical output channel deteriorates. Since components that are not present in light intensity are present in the noise at the time of measurement, the SN ratio (the ratio of noise to a signal, where a higher value means less noise included in the signal) of the measurement result at an optical output channel decreases with a greater distance between an optical input channel and the optical output channel. When the SN ratio of the measurement result decreases, the accuracy of the reconstruction process (absorption coefficient estimation process) performed based on the measurement result also decreases, and thus the image quality of the images representing the measurement results also deteriorates.

In other words, as shown in FIG. 5B, even when the tumor is located within the range of region 2, the optical-combined imaging apparatus 1000 in the aforementioned Patent Literature 1 performs reconstruction on a fixed imaging subject region that includes regions other than region 2 such as region 1, and so on, by using a measurement result having a low SN ratio, such as the measurement result between the optical input channel In1 and the optical output channel Ou1, and so on. Accordingly, the image quality of the reconstruction result deteriorates. Furthermore, since reconstruction is performed including the measurement results from channel pairs that are not required for reconstruction, the time for imaging becomes long.

Consequently, the present invention is conceived in view of the above-described problem and has as an object to provide an optical-combined imaging method which improves image quality and shortens the time for imaging.

### Solution to Problem

In order to achieve the aforementioned object, the optical-combined imaging method in an aspect of the present invention is an optical-combined imaging method which is a combination of an imaging method and a structure identification method, the imaging method (i) measuring diffused light which is near infrared light emitted onto body tissue and diffusing within the body tissue and (ii) imaging an optical characteristic within the body tissue, and the structure identification method identifying a structural characteristic within the body tissue by performing measurement that is different from the measurement of diffused light, the optical-combined imaging method including: identifying a location and a size of an observation target object that is present within the body tissue, using the structure identification method; determining an imaging subject region including the observation target object, based on the location and the size identified in the identifying; measuring the diffused light propagated through the imaging subject region determined in the determining; estimating the optical characteristic within the imaging subject region based on a result of the measurement in the measuring of the diffused light, and imaging the estimated optical characteristic; and displaying the optical characteristic imaged in the imaging.

Accordingly, for example, an imaging subject region including the observation target object is determined based on the location and size of the observation target object identified by the structure identification method using ultrasound, and imaging by diffuse optical tomography is performed on the imaging subject region. Therefore, there is no need to set a wide fixed imaging subject region in advance so as to be able to cope with all locations and sizes of an observation target object as in the conventional techniques, and in the optical-combined imaging method according to an aspect of the present invention, the imaging subject region can be set narrowly to an appropriate size in accordance with the observation target object. As a result, it is possible to prevent the estimation of an optical characteristic (for example, absorption coefficients), that is, the performance of reconstruction of optical characteristics, based on measurement results of diffused light having a low SN ratio, and thus it is possible to improve the image quality of the image representing the imaged optical characteristic, that is, the reconstruction result, and shorten the time for imaging (particularly, the time for reconstruction).

Furthermore, the measuring of the diffused light may include: emitting the near infrared light onto the body tissue so that the near infrared light is propagated through the imaging subject region determined in the determining; and measuring, as the diffused light, the near infrared light emitted and propagated through the imaging subject region in the emitting.

Accordingly, since the near infrared light is emitted so as to be propagated through the imaging subject region and such emitted near infrared light is measured as diffused light, the time for measuring the diffused light can be shortened. Specifically, conventionally, near infrared light is emitted so as to be propagated over a predetermined wide region regardless of the location and size of the observation target object, and the emitted near infrared light is measured as the diffused light. However, in the optical-combined imaging method according to an aspect of the present invention, the imaging subject region can be set narrowly to an appropriate size, as described above, and thus the emission range of the near infrared light, in other words, the measurement range can be narrowed down and the time for measuring diffused light can be shortened.

Furthermore, the optical-combined imaging method may further include selecting a channel pair that allows measurement of the diffused light propagated through the imaging subject region determined in the determining, from among channel pairs which are respective combinations of an emission channel for emitting near infrared light and a detection channel for detecting diffused light which is the near infrared light emitted from the corresponding emission channel and diffused within the body tissue, the channel pairs having mutually different regions through which the diffused light is propagated, wherein in the emitting, the near infrared light may be emitted from the emission channel of the channel pair selected in the selecting, and in the measuring of the near infrared light, the diffused light propagated through the imaging subject region may be measured by detecting the diffused light at the detection channel of the channel pair selected in the selecting.

Accordingly, it is possible to improve image quality and shorten the diffused light measuring time by using the optical probe including channel pairs each made up of an emission channel (optical input channel) and a detection channel (optical output channel).

Furthermore, the measuring of the diffused light may include: emitting the near infrared light onto the body tissue so that the near infrared light is propagated through a predetermined first region including the imaging subject region within the body tissue; and measuring, as the diffused light, the near infrared light emitted and propagated through the predetermined first region in the emitting, wherein in the estimating within the imaging subject region, the optical characteristic within the imaging subject region may be estimated based only on a result of the measurement for the diffused light propagated through the imaging subject region, out of a result of the measurement in the measuring of the near infrared light.

For example, the predetermined first region is set fixedly to be wide so as to be able to cope with all locations and sizes of the observation target object. In such a case, even when the near infrared light is emitted so as to be propagated through such first region and then measured as diffused light, in the optical-combined imaging method according to an aspect of the present invention, the optical characteristic is estimated based only on the measurement result corresponding to the diffused light propagated through the imaging subject region, out of the result of the measurement for the first region. As a result, it is possible to prevent the estimation of the optical characteristic based on measurement result of diffused light having a low SN ratio, and thus it is possible to improve the image quality of the image representing the imaged optical characteristic, that is, the reconstruction result.

Furthermore, the optical-combined imaging method may further include selecting a channel pair that allows measurement of the diffused light propagated through the imaging subject region determined in the determining, from among channel pairs which are respective combinations of an emission channel for emitting near infrared light and a detection channel for detecting diffused light which is the near infrared light emitted from the corresponding emission channel and diffused within the body tissue, the channel pairs having mutually different regions through which the diffused light is propagated, wherein in the emitting, the near infrared light may be emitted from the respective emission channels of the channel pairs, in the measuring of the near infrared light, the diffused light propagated through the predetermined first region may measured by detecting the diffused light at the respective detection channels of the channel pairs, and in the estimating within the imaging subject region, the optical characteristic within the imaging subject region may be estimated based only on a result of the measurement using the channel pair selected in the selecting, out of the result of the measurement in the measuring of the near infrared light.

Accordingly, image quality can be improved using the optical probe including channel pairs each made up of an emission channel (optical input channel) and a detection channel (optical output channel).

Furthermore, the optical-combined imaging method may further include: judging whether or not the observation target object is present within the body tissue, using the structure identification method; measuring the diffused light propagated through a predetermined second region within the body tissue, when it is judged in the judging that the observation target object is not present; estimating the optical characteristic within the predetermined second region based on a result of the measurement in the measuring of the diffused light propagated through the predetermined second region, and imaging the estimated optical characteristic; and displaying the optical characteristic imaged in the estimating within the predetermined second region, wherein in the identifying, the location and the size of the observation target object may be identified when it is judged in the judging that the observation target object is present.

For example, the predetermined second region is set fixedly to be wide. Therefore, in the optical-combined imaging method according to an aspect of the present invention, when the observation target object cannot be verified within the body tissue, imaging using diffuse optical tomography can be performed over a wide range and the condition of the body tissue can be recognized, without suspending the imaging.

Furthermore, the optical-combined imaging method may further include: identifying a biological characteristic of the observation target object based on the optical characteristic estimated in the estimating within the imaging subject region, and generating diagnosis supplementary information indicating the biological characteristic, wherein in the displaying, the diagnosis supplementary information may be further displayed.

For example, when the observation target object is a tumor, whether the tumor is malignant or benign is identified as a biological characteristic and displayed, and thus it is possible to provide a diagnosis that is useful to the test subject.

Furthermore, the optical-combined imaging method may further include: identifying a functional characteristic within the body tissue by performing the measurement that is different from the measurement of diffused light, and generating functional information indicating the functional characteristic, wherein in the displaying, the functional information may be further displayed.

For example, by measuring ultrasound emitted onto the body tissue, blood flow volume, and so on, within the body tissue is identified as a functional characteristic and displayed, and thus it is possible to provide a diagnosis that is useful to the test subject.

It should be noted that the present invention can be realized not only as such an optical-combined imaging method, but also as an apparatus and integrated circuit that perform imaging according to such method, a program for causing a computer to execute imaging according to such method, and a recoding medium on which such program is stored.

### Advantageous Effects of Invention

The optical-combined imaging method according to the present invention can improve image quality and shorten the time for imaging. Specifically, the optical-combined imaging method according to the present invention can minimize the use of measurement results having low SN ratio and, as a result, realize an improvement in image quality of the reconstruction result and a reduction in the amount of processing as well as processing time involved in image reconstruction.

### BRIEF DESCRIPTION OF DRAWINGS

FIG. 1 is a schematic diagram showing a configuration example for diffuse optical tomography.

FIG. 2 is a block diagram showing a configuration example of an optical-combined imaging apparatus 1000 in the aforementioned Patent Literature 1.

FIG. 3 is flowchart showing operations in optical diffusion tomography in a conventional optical-combined imaging method.

FIG. 4A is a diagram showing an example of an external appearance of a combined probe that combines respective optical and ultrasound probes.

FIG. 4B is a diagram showing an example of light propagation regions between optical input channels and optical output channels.

FIG. 5A is a diagram showing the positional relationship of a tumor with respect to an imaging subject region.

FIG. 5B is a diagram showing the positional relationship between a tumor and light propagation regions in an imaging subject region.

FIG. 6 is an external view of an optical-combined imaging apparatus 100 in Embodiment 1 of the present invention.

FIG. 7 is a block diagram showing a configuration example of the optical-combined imaging apparatus in Embodiment 1.

FIG. 8 is a diagram showing an external appearance of a fixed-type combined probe in Embodiment 1.

FIG. 9 is a flowchart showing an operation of the optical-combined imaging apparatus in Embodiment 1.

FIG. 10 is a flowchart showing processes in step S103 in FIG. 9 in Embodiment 1.

FIG. 11A is a diagram showing the positional relationships of an imageable region, an imaging subject region, and a tumor.

FIG. 11B is a diagram showing the positional relationship between a tumor and light propagation regions in an imageable region and an imaging subject region.

FIG. 12 is flowchart showing an operation of an optical-combined imaging apparatus in Modification 1 of Embodiment 1 of the present invention.

FIG. 13 is a flowchart showing an operation of an optical-combined imaging apparatus in Modification 2 of Embodiment 1.

FIG. 14 is a flowchart showing an operation of an optical-combined imaging apparatus in Modification 3 of Embodiment 1.

FIG. 15 is a flowchart showing an operation of an optical-combined imaging apparatus in Modification 4 of Embodiment 1.

FIG. 16 is a flowchart showing an operation of an optical-combined imaging apparatus in Modification 5 of Embodiment 1.

FIG. 17A is a diagram showing an example of a recording medium which stores a program for implementing an optical-combined imaging method using a computer system, in Embodiment 2 of the present invention.

FIG. 17B is a diagram showing another example of a recording medium which stores a program for implementing an optical-combined imaging method using a computer system, in Embodiment 2.

FIG. 17C is a diagram showing system for implementing an optical-combined imaging method using a computer system, in Embodiment 2.

FIG. 18A is a block diagram showing a configuration of an optical-combined imaging apparatus according to the present invention.

FIG. 18B is a flowchart showing an optical-combined imaging method according to the present invention.

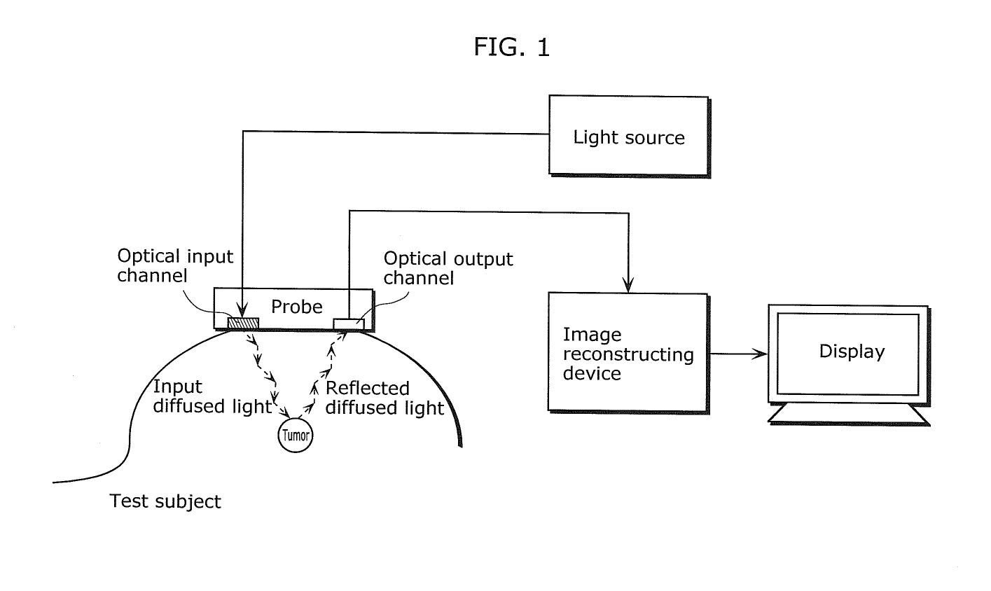

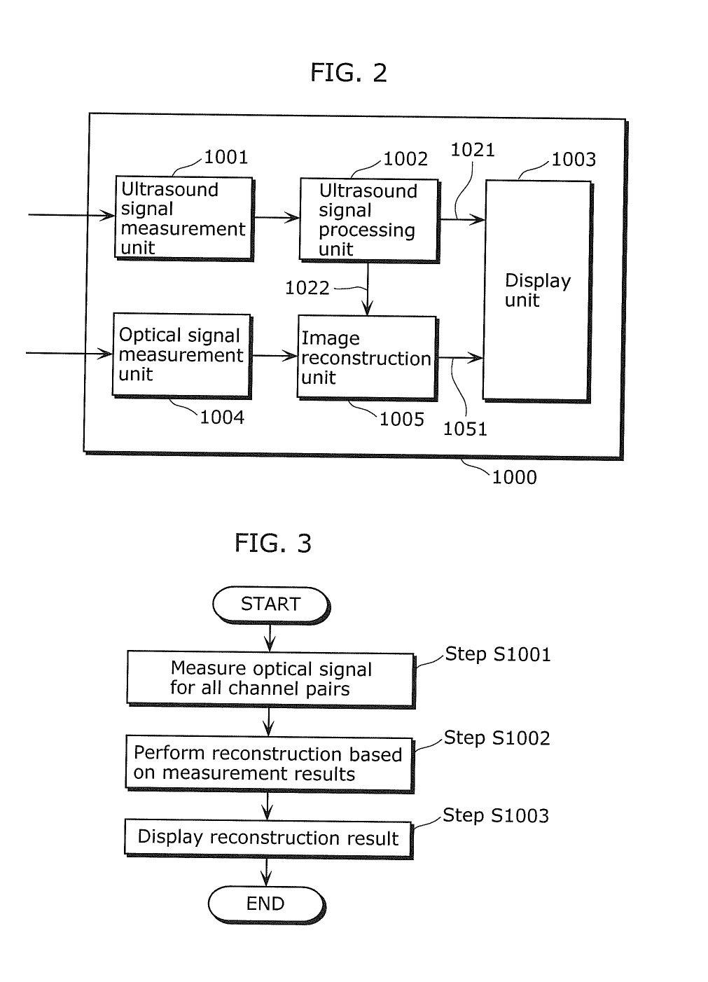

### DESCRIPTION OF EMBODIMENTS

Hereinafter, embodiments of the present invention shall be described with reference to the Drawings.

### Embodiment 1

Embodiment 1 of an optical-combined imaging method and an optical-combined imaging apparatus according to the present embodiment shall be described with reference to the Drawings.

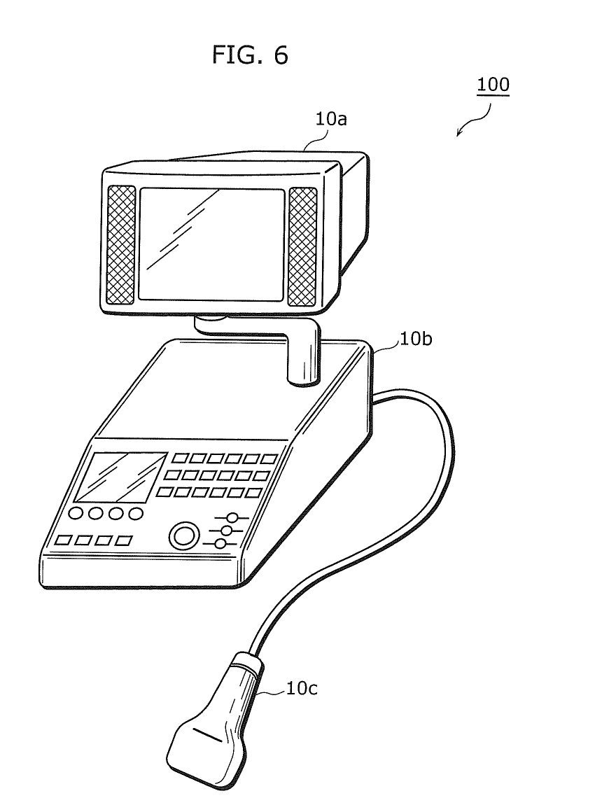

FIG. 6 is an external view of an optical-combined imaging apparatus 100 in the present embodiment.

The optical-combined imaging apparatus 100 in the present embodiment is an imaging apparatus which combines the use of ultrasound and diffuse optical tomography, and can improve image quality and shorten the time for imaging. Such an optical-combined imaging apparatus 100 mainly includes a display device 10a, a main device 10b, and a combined probe 10c.

The display device 10a is a display device using liquid crystal, CRT, and the like, for displaying images obtained using the optical-combined imaging method, medically-necessary information, and so on, and includes a touch panel, or the like, which receives input from an operator.

The main device 10b includes: a transmission and reception circuit for controlling the transmission and reception of ultrasound waves and near infrared light in the combined probe 10c, a signal and image processing circuit including a digital signal processor (DSP), a RAM, and so on, for processing various signals or images; and a switch group which receives operations from the operator.

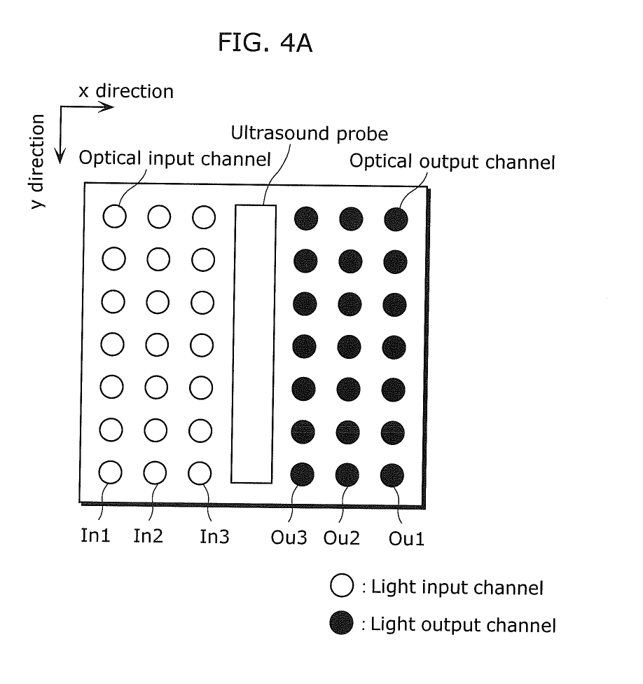

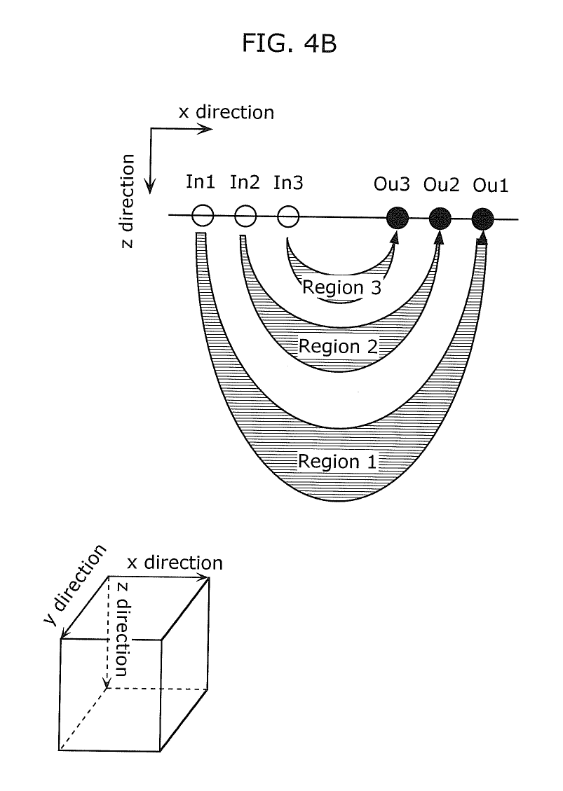

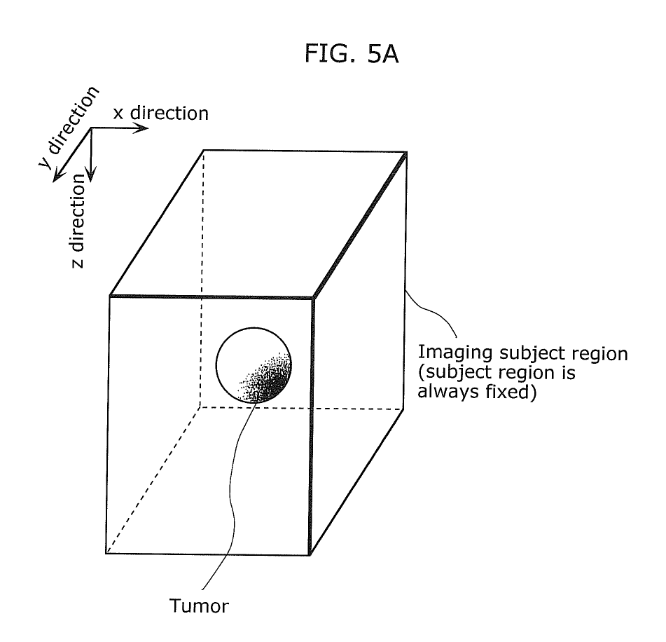

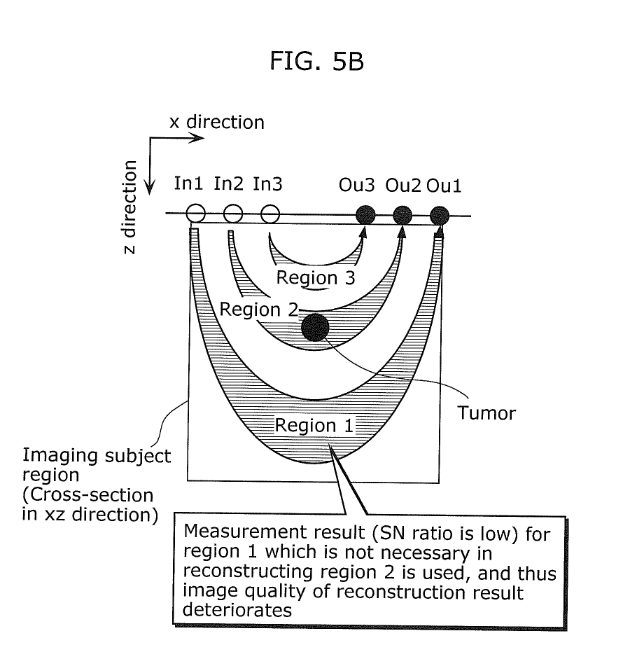

For example, in the same manner as the combined probe shown in FIG. 4A, the combined probe 10c includes: an ultrasound probe including an ultrasound transducer, an acoustic lens, and so on, for transmitting and receiving ultrasound waves; and an optical probe including optical input channels (emission channels) and optical output channels (detection channels). For example, the ultrasound probe is provided in between the optical input channels and the optical output channels. It should be noted that a channel pair is configured from a pairing of an optical input channel and an optical output channel.

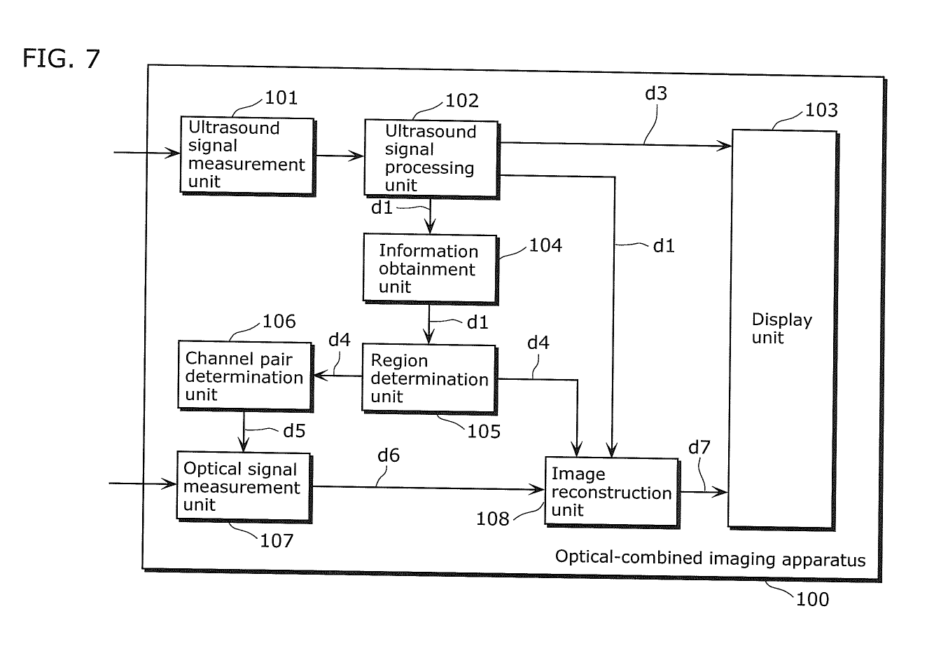

FIG. 7 is a block diagram showing a configuration example of the optical-combined imaging apparatus 100 in the present embodiment.

The optical-combined imaging apparatus 100 includes an ultrasound signal measurement unit 101, an ultrasound signal processing unit 102, a display unit 103, an information obtainment unit 104, a region determination unit 105, a channel pair determination unit 106, an optical signal measurement unit 107, and an image reconstruction unit 108. It should be noted that the ultrasound signal measurement unit 101 and the optical signal measurement unit 107 make up the combined probe 10c, the ultrasound signal measurement unit 101 corresponds to the ultrasound probe and the optical signal measurement unit 107 corresponds to the optical probe. In addition, the display unit 103 corresponds to the display device 10a.

The ultrasound signal measurement unit 101 and an ultrasound signal processing unit 102 process ultrasound signals. The ultrasound signal measurement unit 101 emits ultrasound waves, then detects and measures, as an ultrasound signal, the ultrasound waves reflected and scattered within the living body. The ultrasound signal processing unit 102 analyzes and images the ultrasound signal measured by the ultrasound signal measurement unit 101, and outputs, to the display unit 103, ultrasound image data d3 representing an image obtained from the analysis. In addition, the ultrasound signal processing unit 102 identifies the location, shape, size, and so on, of a tumor through the above-described analysis, and outputs, to the information obtainment unit 104 and the image reconstruction unit 108, tumor information d1 indicating such location, shape, size, and so on. The tumor information d1 obtained by the ultrasound signal processing unit 102 may be two-dimensional information or three-dimensional information. When the tumor information d1 is two-dimensional information, the obtainable information indicating the shape of the tumor is information indicating a specific cross-sectional shape of the tumor, and the three-dimensional shape, size, and so on, of the tumor is estimated separately from such information indicating the specific cross-sectional shape.

The information obtainment unit 104, the region determination unit 105, the channel pair determination unit 106, the optical signal measurement unit 107, and the image reconstruction unit 108 execute processes in the diffuse optical tomography.

The information obtainment unit 104 obtains the tumor information d1 from the ultrasound signal processing unit 102 and outputs the tumor information d1 to the region determination unit 105.

The region determination unit 105 obtains the tumor information d1 from information obtainment unit 104, and determines the region to be imaged (imaging subject region) from within a predetermined imageable region, based on the tumor information d1. In addition, the region determination unit 105 outputs, to the channel pair determination unit 106 and the image reconstruction unit 108, region information d4 which indicates such imaging subject region.

Upon obtaining the region information d4 from the region determination unit 105, the channel pair determination unit 106 determines (selects), from among the channel pairs included in the optical signal measurement unit 107, at least one valid channel pair having, as a light propagation region, all or part of the imaging subject region indicated by the region information d4. It is to be noted that a channel pair is a pairing of one optical input channel and one optical output channel. In addition, the channel pair determination unit 106 outputs, to the optical signal measurement unit 107, pair information d5 indicating the determined channel pair.

Upon obtaining the pair information d5 from the channel pair determination unit 106, the optical signal measurement unit 107 selects the channel pair indicated by the pair information d5 from among the channel pairs. In addition, the optical signal measurement unit 107 emits near infrared light from the optical input channel included in the selected channel pair, then, using the optical output channel included in the selected channel pair, detects and measures, as an optical signal, the near infrared light (diffused light) that has diffused within the living body. The optical signal measurement unit 107 outputs optical measurement information d6 indicating the measurement result to the image reconstruction unit 108. It should be noted that, when plural channel pairs are indicated by the pair information d5, the optical signal measurement unit 107 repeatedly performs the above-described optical signal measurement sequentially for each of the channel pairs indicated by the pair information d5.

The image reconstruction unit 108 obtains the optical measurement information d6 outputted by the optical signal measurement unit 107, the region information d4 outputted by the region determination unit 105, and the tumor information d1 outputted by the ultrasound signal processing unit 102. In addition, the image reconstruction unit 108 estimates, in other words reconstructs, the absorption coefficients (or, both the absorption coefficients and the scattering coefficients) of the tumor portion in the imaging subject region, by using the optical measurement information d6, the region information d4, and the tumor information d1. At this time, the image reconstruction unit 108 performs the reconstruction using diffusion approximation. The image reconstruction unit 108 images the absorption coefficients obtained through the reconstruction, and outputs, to the display unit 103, optical image data d7 representing the image (reconstruction result). More specifically, in the present embodiment, the image reconstruction unit 108 includes a display control unit which controls the display unit 103.

The display unit 103 displays images respectively represented by the ultrasound image data d3 and the optical image data d7.

It should be noted that although in the present embodiment the combined probe 10c including the optical signal measurement unit 107 and the ultrasound signal measurement unit 101 is of a scanner type that measures while being moved by the user as shown in FIG. 6, the combined probe 10c may be of a type that performs the measurement in a fixed state (fixed type). For example, the fixed-type combined probe 10c may be formed in a dome shape which covers a breast for breast cancer diagnosis.

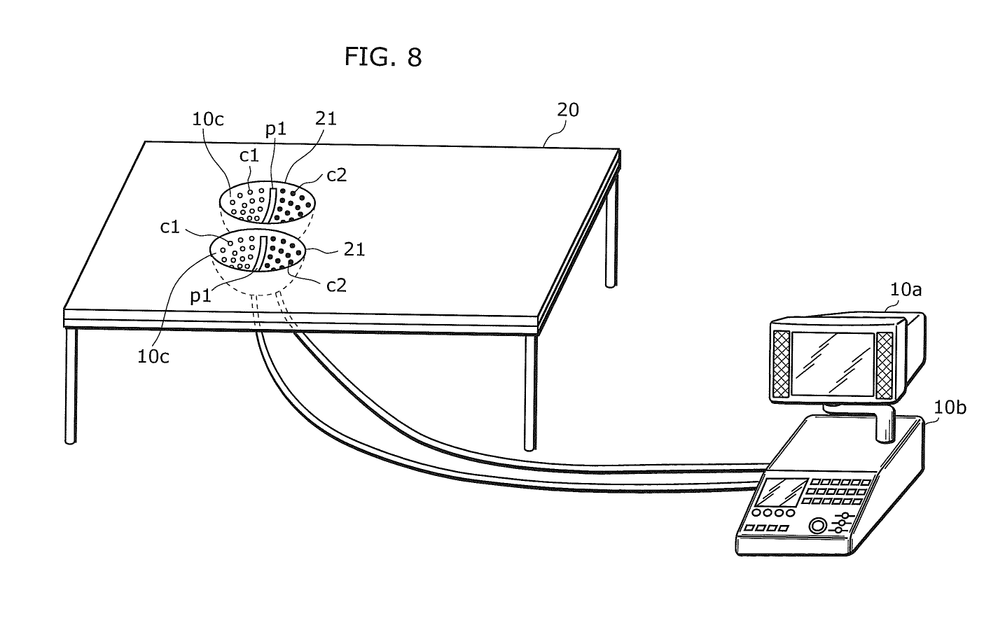

FIG. 8 is a diagram showing an external appearance of the fixed-type combined probe 10c.

As described above, the fixed-type combined probe 10c is formed in a dome shape. The inner surface of the concave part of the combined probe 10c is provided with a previously described ultrasound probe p1, and an optical probe including channel pairs each made up of an optical input channel c1 and an optical output channel c2.

For example, two of such fixed-type combined probes 10c are affixed to a diagnostic bed 20. Two holes are provided in the diagnostic bed 20, and each of the fixed-type combination probes 10c is fitted and affixed in a corresponding one of the holes 21 such that the inner surface of the concave part faces upward. A female test subject lies face-down on the bed 20 and inserts her breasts in the concave part of the combined probe 10c. In this manner, measurement is performed in a state in which the breasts are set in the concave part of the combined probe 10c.

It should be noted that the ultrasound probe and the diffuse optical tomography optical probe may be provided in the same housing, and may each be provided in independent housings. However, when the diffuse optical tomography optical probe is of the scanner type, it is preferable that both probes be provided within the same housing in view of the obtainability of the correspondence relationship between the diffuse optical tomography and ultrasound imaging regions.

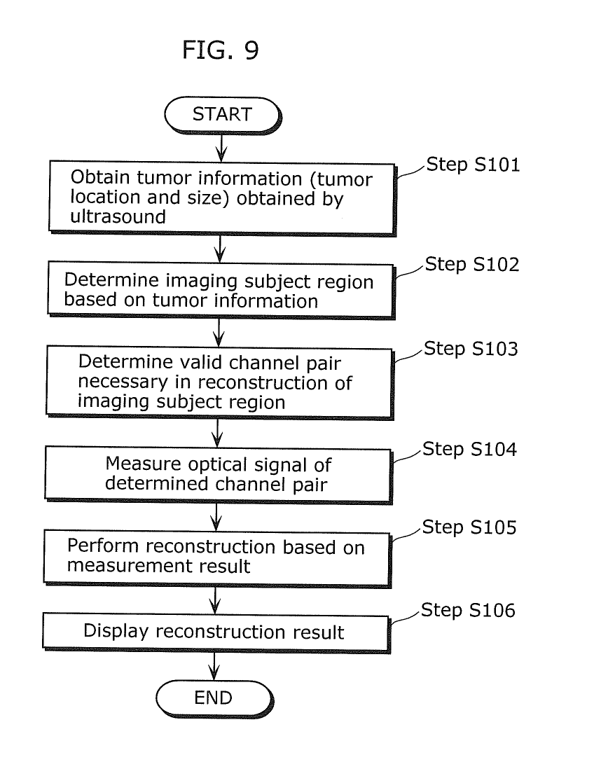

FIG. 9 is a flowchart showing an operation of the optical-combined imaging apparatus 100 in the present embodiment. It should be noted that, here, description shall be made with reference to FIG. 9 and centering on the operation of the diffuse optical tomography portion of the optical-combined imaging apparatus 100 according to the present invention.

First, the information obtainment unit 104 obtains the tumor information d1 obtained using ultrasound (step S101). Next, the region determination unit 105 automatically determines the imaging subject region based on the obtained tumor information d1 so that at least the tumor area is included (step S102). The imaging subject region is determined so as to fit within an imageable region that allows reconstruction using the optical input channels and the optical output channels provided in the optical signal measurement unit 107.

It should be noted that the tumor information which indicates the size, location, and so on, of the tumor may be determined manually while the user checks the tumor location in the ultrasound image (image represented by the ultrasound image data d3), and may be automatically determined through comparison, and so on, with an image feature amount or a past case database, and the like. Furthermore, the imaging subject region may also be manually determined while the user checks the tumor location using the ultrasound image. Furthermore, due to constraints, and the like, on the size of the combined probe 10c, there are cases where it is difficult to place the optical input channels and optical output channels to have equal sensitivity throughout all regions of the imaging subject region. In this case, the optical signal measurement unit 107 is designed, for example, so that sensitivity increases in the central part of the combination probe 10c in a cross-section parallel to the xy plane in FIG. 4A. In such a case, it is preferable that the tumor location be in the part with high sensitivity. Therefore, the optical-combined imaging apparatus 100 may include an interface such as a display unit which prompts the user to move the combined probe 10c so that the tumor location settles within a predetermined region. For example, the optical-combined imaging apparatus 100 may display information indicating the high-sensitivity region within the imageable region by superimposition on the ultrasound image, and thus indicate the positional correlation between the tumor location indicated by the ultrasound image and the high-sensitivity region in the imageable region.

Next, the channel pair determination unit 106 determines the valid channel pair necessary for the reconstruction of the imaging subject region (step S103). The optical signal measurement unit 107, using the valid channel pair determined for use in step S103, emits a laser beam (near infrared light) from the optical input channel, and measures, as an optical signal, the diffused light that has been propagated within the living body and has reached the optical output channel (step S104). The image reconstruction unit 108 reconstructs (estimates) the absorption coefficient of each voxel within the imaging subject region determined in step S102, based on the result of the measurement in step S104, and images the reconstruction result (step S105). In the reconstruction of the absorption coefficients, the image reconstruction unit 108 calculates such absorption coefficients using iterative computation. At this time, the image reconstruction unit 108 determines the initial value of the absorption coefficient based on the tumor information d1. Here, in solving the inverse problem at the time of reconstruction in diffuse optical tomography, the use of iterative computation is preferable from the perspective of reconstruction accuracy. However, the reconstruction result (absorption coefficient) may be obtained directly without iterative computation, for example, by calculating a pseudo inverse matrix of the matrix representing a model such as diffusion approximation. It should be noted that in directly obtaining the reconstruction result, the setting of the initial value in step S105 is unnecessary. It should be noted that the absorption coefficient may be an absolute value, and may be a relative value indicating the difference from a reference value. Furthermore, the target of reconstruction is not limited to absorption coefficients, and may be a feature amount indicating other optical characteristics such as scattering coefficients.

Lastly, the display unit 103 displays the imaged reconstruction result (estimated absorption coefficients) (step S106). The imaged reconstruction result may two-dimensionally display a cross-section of the imaging subject region, and may three dimensionally display the entire imaging subject region. Here, when the reconstruction result of the diffuse optical tomography is to be displayed by superimposition on the ultrasound image, displaying in two dimensions or displaying in three dimensions is made uniform between the two.

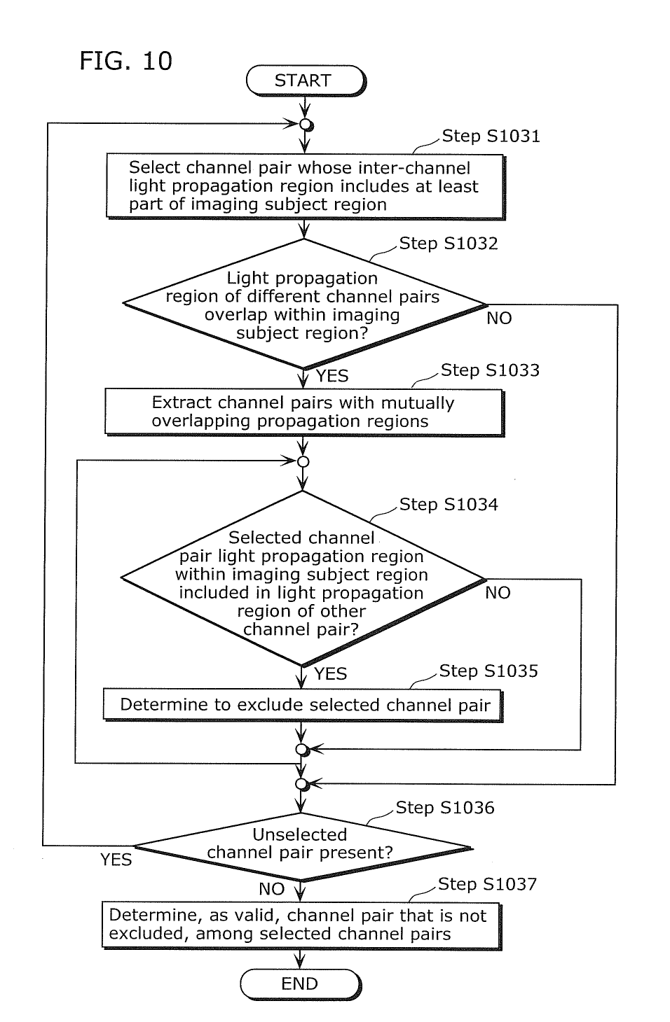

FIG. 10 is a flowchart showing the processes in step S103.

First, the channel pair determination unit 106 selects a channel pair in which the light propagation region between the channels includes at least part of the imaging subject region (step S1031). Here, the method of determining the light propagation region shall be discussed in detail. The measurement value of an optical output channel is determined based on an optical characteristic such as the absorption coefficient or scattering coefficient in each voxel (respective three-dimensional spaces after segmentation, when a three dimensional space is segmented into minute three-dimensional spaces. For example, in the case of segmentation into plural cubes, each of the cubes becomes a voxel). Here, voxel sensitivity is defined as in (Expression 1).

$$\begin{matrix}( Expression 1 ) & \\ J ( x , y , z ) = \frac{\partial {m}_{SD}}{\partial {μ}_{x , y , z}} & [ Math 1 ]\end{matrix}$$

In (Expression 1) m_{SD} represents a logical measurement value of an optical output channel D with respect to an optical input channel 5, and μ_{x, y, z} represents the optical characteristic of a voxel located in a three-dimensional coordinate (x, y, z) within the imaging subject region. J(x, y, z) is the sensitivity of the voxel located in (x, y, z) and indicates the percentage of the amount of change in the measurement value of the optical output channel D with respect to the amount of change in the optical characteristic. Here, each amount of change is the amount of change from a reference value. Because the amount of light in each voxel and the sensitivity of the voxel are in a proportional relationship, the light propagation region is defined as the region in which the sensitivity of the voxel is equal to or greater than a predetermined threshold. Voxel sensitivity is largely dependent on voxel depth in particular, and sensitivity decreases with an increase in depth. Therefore, the threshold in determining the light propagation region may be switched depending on the depth of the voxel, and the threshold may be made lower for a voxel that is in a deeper location. Furthermore, selection may be performed based on a predetermined condition such as selecting only when the light propagation region includes a region that is equal to or greater than a fixed volume within the imaging subject region.

Next, the channel pair determination unit 106 judges whether or not there is another channel pair having a propagation region that overlaps with the light propagation region of the channel pair selected in step S1031, in the imaging subject region (step S1032). Here, when the channel pair determination unit 106 judges that there is another channel pair (YES in step S1032), the channel pair determination unit 106 extracts the other channel pair having a propagation region that overlaps with the channel pair selected in S1031 (step S1033). At this time, when there are plural other channel pairs, the channel pair determination unit 106 extracts all of the other channel pairs. On the other hand, when the channel pair determination unit 106 judges that there is no other channel pair (NO in step S1032), the channel pair determination unit 106 executes the process in step S1036 to be described later.

It should be noted that it is acceptable to make a judgment of overlapping only when the volume of the overlap portion or the percentage of the light propagation region accounted for by the overlap portion within the imaging subject region, and the like, is equal to or greater than a predetermined threshold.

Next, the channel pair determination unit 106 judges whether or not the light propagation region of the selected channel pair that is within the imaging subject region is included in the light propagation region of the one or more other channel pairs extracted in step S1033 (step S1034). Here, when the channel pair determination unit 106 judges that the light propagation region is included (YES in S1034), the channel pair determination unit 106 determines that the channel pair selected in step S1031 is to be excluded from the channel pairs to be used in measurement (step S1035).

It should be noted that even when the light propagation region is not completely included within the light propagation region of the other channel pair, whether or not to exclude the selected channel pair from the channel pairs to be used in the measurement may be determined based on, for example, the volume of the region that is not included, the size of a predetermined cross-section in the region that is not included (for example, the length of a segment having a maximum or minimum length, and so on, among line segments that can be drawn within a cross-section that is parallel to the xy plane of the region that is not included), or the percentage of the light propagation region accounted for by the region that is not included, or location information such as whether the region that is not included is in the central part or the periphery of the imaging subject region. In addition, whether or not a region that is not included in the propagation region of any channel pair, within the imaging subject region, is equal to or lower than a predetermined percentage may be adopted as a judgment condition.

The processes in step S1034 and step S1035 are repeated for each of the channel pairs extracted in step S1033. Several methods are possible for the method of selecting a channel pair in step S1031. For example, by selecting in sequence from a channel pair with the greatest distance between channels, channel pairs having greater inter-channel distance are preferentially excluded.

Next, the channel pair determination unit 106 judges whether or not there is an unselected channel pair having a light propagation region that includes at least a part of the imaging subject region (step S1036), and repeats the processes from step S1031 when it judges that there is an unselected pair (YES in step S1036). On the other hand, when it is judged that there is no unselected channel pair (NO in step S1036), the region determination unit 105 determines the channel pair selected in step S1031 and not excluded in step S1035 to be a valid channel pair that is to be used in the measurement (step S1037).

Here, in the case where light propagation regions of channel pairs having similar inter-channel distances in particular overlap, there are instances where the use of channel pairs having overlapping light propagation regions enhances the SN ratio of the optical signal. For example, assuming that the light propagation regions of the three channel pairs channel pair A, channel pair B, and channel pair C overlap, and the SN ratio of the channel pair A is lower than those of the channel pair B and the channel pair C due to the contact condition, and so on, of the combined probe 10c. In this case, the SN ratio of the optical signal improves more by using the three channel pairs together than by using only the channel pair A. Therefore, channel pairs having overlapping light propagation regions may be allowed, and so on, up to a predetermined number. Furthermore, there are instances when measurement is performed on a per channel pair basis, and the measurement of the diffused light for each channel pair requires a time of a few hundred milliseconds or more. In particular, when processing that is close to real-time is required, the measurement time can become a bottleneck. As such, the condition for allowing overlapping channel pairs may be set based on the measurement time.

Although the channel pairs to be used is dynamically determined here according to the imaging subject region, channel pairs to be used for a predetermined imaging subject region may be determined in advance and the results thereof held. For example, the imageable region is segmented into N-regions in the depth direction, and the channel pair to be used for each region resulting from the segmentation (segment region) is stored in advance in a memory. At this time, in step S103, the channel pair determination unit 106 determines the segment region within the imageable region to which the imaging subject region corresponds, and determines the valid channel pair required for the reconstruction by reading, from the memory, the channel pair to be used for such segment region. When the imaging subject region is spread over plural segment regions, the channel pair determination unit 106 determines all of the channel pairs necessary for each segment region as the valid channel pairs required for the reconstruction. Alternatively, the channel pair determination unit 106 may select segment regions in such a way that the percentage of a region, out of the imaging subject region, that is included in any of the segment regions is equal to or greater than a threshold, and determine the channel pairs that are required for the selected segment region. In addition, at this time, the channel pair determination unit 106 may set an area-based condition, such as, the central part of the imaging subject region must necessarily be included in any of the segment regions.

Furthermore, in step S103, the voxel size may be adaptively determined based on the tumor information or the size of the imaging subject region. For example, the voxel size is adjusted to correspond to the tumor size. Alternatively, since the amount of computational processing in the reconstruction process increases following an increase in the number of voxels, the voxel size is set so that the number of voxels included in the imaging subject region is equal to or less than a predetermined number. In addition, when the SN ratio of the measurement result decreases and the reconstruction accuracy deteriorates as the region to be imaged becomes deeper, the detection sensitivity and resolution for a deep region decreases compared to a shallow region. Therefore, as the imaging subject region becomes deeper the voxel size may be enlarged or the minimum value that can be set for the voxel size may be increased.

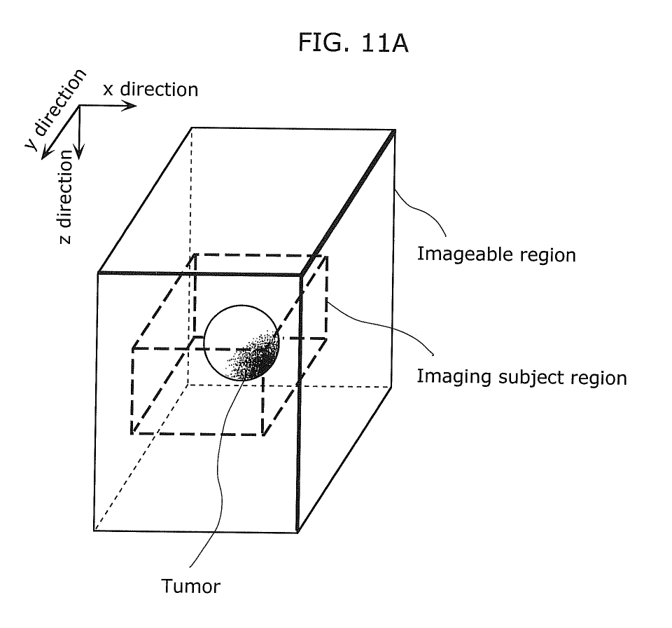

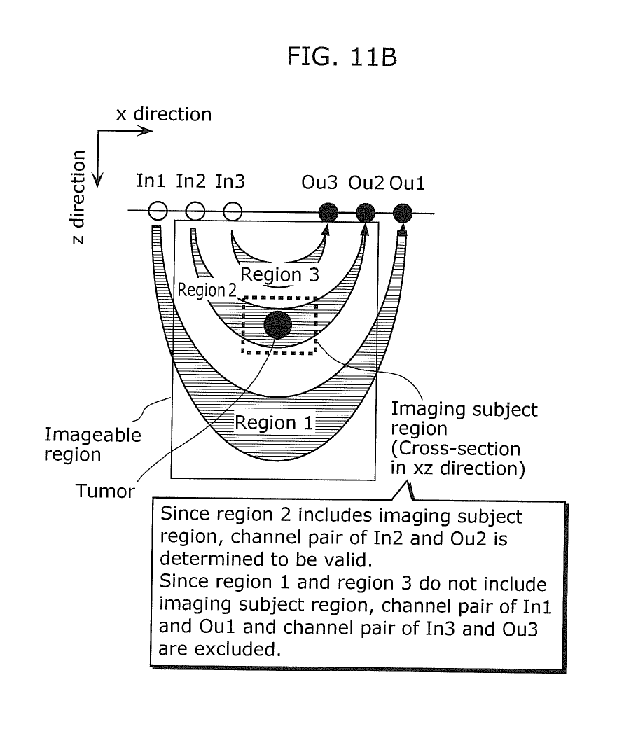

FIG. 11A is a diagram showing the positional relationships of an imageable region, an imaging subject region, and a tumor. The optical-combined imaging apparatus 100 determines the imaging subject region out of a predetermined imageable region in step S102 in FIG. 9. The imaging subject region (the region surrounded by the bold dotted lines in FIG. 11A) is determined in such a way that the tumor is included in the imaging subject region. Here, the imageable region is the region surrounded by the solid lines, and it can be seen that the imaging subject region is set with the region being limited to within the imageable region. It should be noted that when displaying the diffuse optical tomography image (the image represented by the optical image data d7) by superimposition on the on the ultrasound image, the display unit 103 may, in order to visually notify the user of the imaging subject region of the diffuse optical tomography inside the ultrasound image, display the border of the imaging subject region.

FIG. 11B is a diagram showing the positional relationship between a tumor and light propagation regions in an imaging subject region. The optical-combined imaging apparatus 100 determines a valid channel pair required for the reconstruction of the imaging subject region, in step S103 in FIG. 9. Specifically, among the channel pair (hereinafter called the first channel pair) consisting of the optical input channel In1 and the optical output channel Ou1, the channel pair (hereinafter called the second channel pair) consisting of the optical input channel In2 and the optical output channel Ou2, and the channel pair (hereinafter called the third channel pair) consisting of the optical input channel In3 and the optical output channel Ou3, only the second channel pair includes the imaging subject region. Therefore, the optical-combined imaging apparatus 100 determines the second channel pair as the valid channel pair, and performs reconstruction using only the measurement result from the second channel pair. In other words, the optical-combined imaging apparatus 100 excludes the first channel pair and the second channel pair, and does not perform reconstruction using the measurement results from those pairs. It should be noted that other channel pairs, such as a channel pair consisting of the optical input channel In1 and the optical output channel Ou2, are not illustrated.

In such manner, in the present embodiment, the location and size of the tumor is identified, an imaging subject region including the tumor is determined from the imageable region, measurement of diffused light is performed only on the imaging subject region, and imaging is performed based on the result of the measurement. Therefore, compared to the conventional method in which a predetermined imageable region is treated as the imaging subject region, and measurement of diffused light and imaging are performed on the entirety of the imageable region, in the present embodiment, it is possible to omit (i) the measurement of diffused light that is not propagated through the imaging subject region and has a low SN ratio and (ii) the estimation of absorption coefficients based on the result of such measurement, and thus it is possible to improve the image quality of the images representing the estimated absorption coefficients. In addition, the time for imaging can be shortened.

(Modification 1)

Hereinafter, a first modification of the optical-combined imaging apparatus 100 in the present embodiment shall be described.

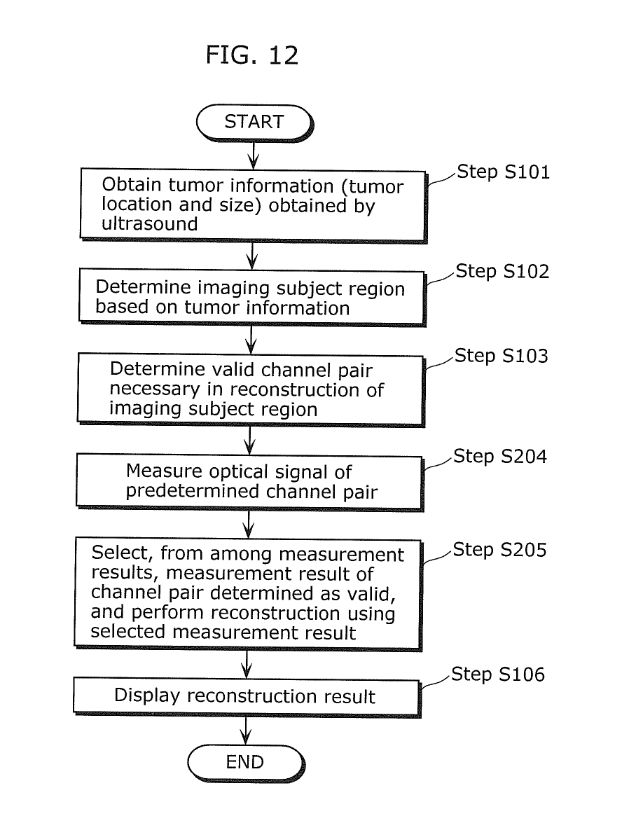

FIG. 12 is a flowchart showing an operation (particularly, an operation of the diffuse optical tomography part) of the optical-combined imaging apparatus 100 in the present modification. It should be noted that, compared to the processes shown in the flowchart in FIG. 9 in the above-described embodiment, the processes in the present modification are different in that the channel pair which measures diffused light is fixed.

First, in the same manner as in the above-described embodiment, the information obtainment unit 104 obtains the tumor information d1 obtained using ultrasound (step S101). Next, the region determination unit 105 automatically determines, based on the obtained tumor information d1, the imaging subject region so that at least the tumor area is included (step S102). Next, the channel pair determination unit 106 determines the valid channel pair necessary for the reconstruction of the imaging subject region (step S103).

Here, in the present modification, regardless of the determination result in step S103, the optical signal measurement unit 107 performs measurement using a predetermined channel pair, that is, channel pairs that have been previously set (S204). Here, the previously-set channel pairs are assumed to be channel pairs including the channel pair determined in step S103. For example, the previously-set channel pairs are all the channel pairs included in the combined probe 10c, and the light propagation regions of such channel pairs also include propagation regions that do not overlap with the imaging subject region determined in step S102. Furthermore, for example, all the regions within the imageable region overlap with any one of the light propagation regions of the previously-set channel pairs.

The image reconstruction unit 108 selects, from among the measurement results in step S204, the measurement result of the valid channel pair determined for use in step S103, and performs reconstruction using the selected measurement result (step S205). In addition, the display unit 103 displays the imaged reconstruction result (estimated absorption coefficients) (step S106).

It should be noted that, in the reconstruction process, the level-of-contribution-to-reconstruction of the measurement result of each of the channel pairs may be determined as a weight, and reconstruction may be performed with weights being assigned. The weight is determined with respect to a channel pair or the light propagation region of such channel pair, based on the same index as that at the time of channel pair selection in step S103, such as the volume of the overlap portion between the light propagation region and the imaging subject region for the channel pair. For example, the bigger the volume of the overlap portion of a light propagation region, the greater the weight that is determined for that propagation region. Since the data necessary for assigning weights can be obtained in step S103, processing up to the assigning of weights may be performed in step S103.

In addition, it is also possible to assign weights in voxel-units which is a finer grading than the channel pair (light propagation region). In the reconstruction process using iterative computation, an evaluation function is defined, and processing is repeated until convergence to the value of the evaluation function is recognized. At this time, the evaluation function includes a term called an error term shown in (Expression 2) below.

$$\begin{matrix}( Expression 2 ) & \\ A = {\sum }_{allSD} {\sum }_{allVOXEL} ( J ( x , y , z ) \times \partial {μ}_{x , y , z} ) - \partial {M}_{SD} & [ Math 2 ]\end{matrix}$$

In (Expression 2), allSD represents all the channel pairs to be used, allVOXEL represents all the voxels included in the propagation region of each channel pair. M_{SD} represents the actual measurement, at the optical output channel D, of the light inputted from the optical input channel S. The expression

[Math 3]

∂M_{SD} (Expression 3)

in (Expression 2) represents the amount of change in measurement value between reference data and an actual measurement subject. On the other hand, the expression

[Math 4]

J(x,y,z)×∂μ_{x,y,z} (Expression 4)

in (Expression 2) corresponds to

[Math 5]

∂m_{SD} (Expression 5)

in (Expression 1), and represents the logical value of the amount of change in measurement value at the optical output channel D. Therefore, error term A is the result obtained by adding, for each channel pair, the difference between the logical value and the actual measurement value to all the channel pairs. At the time of reconstruction, the value of

[Math 6]

∂μ_{x,y,z} (Expression 6)

which is an unknown parameter in (Expression 2), is sequentially updated so that the value of the evaluation function including the error term A becomes smaller.

Here, since J(x, y, z) is voxel sensitivity, the level of contribution of each voxel with respect to the evaluation function value differs depending on sensitivity, and the level of contribution is higher for a voxel having a higher sensitivity. For example, assume that the value of error term A is to be changed by 0.1. When a voxel has a sensitivity of 1, it would be sufficient to change the absorption coefficient by only 0.1. However, in the case of a voxel having a sensitivity of 0.1, the absorption coefficient must be changed by 1. In this manner, in the reconstruction process, there is a tendency to preferentially update the absorption coefficient of a voxel having a high sensitivity. Therefore, weights may be assigned to each voxel for the evaluation function so that voxels having differing sensitivities have equal levels of contribution with respect to the evaluation function value. The determination of the weights can be performed based on a reciprocal of sensitivity, or further simplified so as to depend on the depth of the voxel, such as increasing the weight for a voxel with a deeper location.

In such manner, in the present modification, the location and size of the tumor is identified, an imaging subject region including the tumor is determined from the imageable region, and measurement of diffused light is performed with respect to a predetermined region (for example, the imageable region) including the imaging subject region. In addition, in the present modification, imaging is performed based only on the measurement results for the imaging subject region, out of the measurement results for the predetermined region. Therefore, compared to the conventional method in which a predetermined region is treated as the imaging subject region, and measurement of diffused light and imaging are performed on the entirety of the imaging subject region, in the present modification, it is possible to omit the estimation of absorption coefficients based on the measurement result for diffused light that is not propagated through the imaging subject region and has a low SN ratio, and thus it is possible to improve the image quality of images representing the estimated absorption coefficients. In addition, the time for imaging can be shortened.

(Modification 2)

Hereinafter, a second modification of the optical-combined imaging apparatus 100 in the present embodiment shall be described.

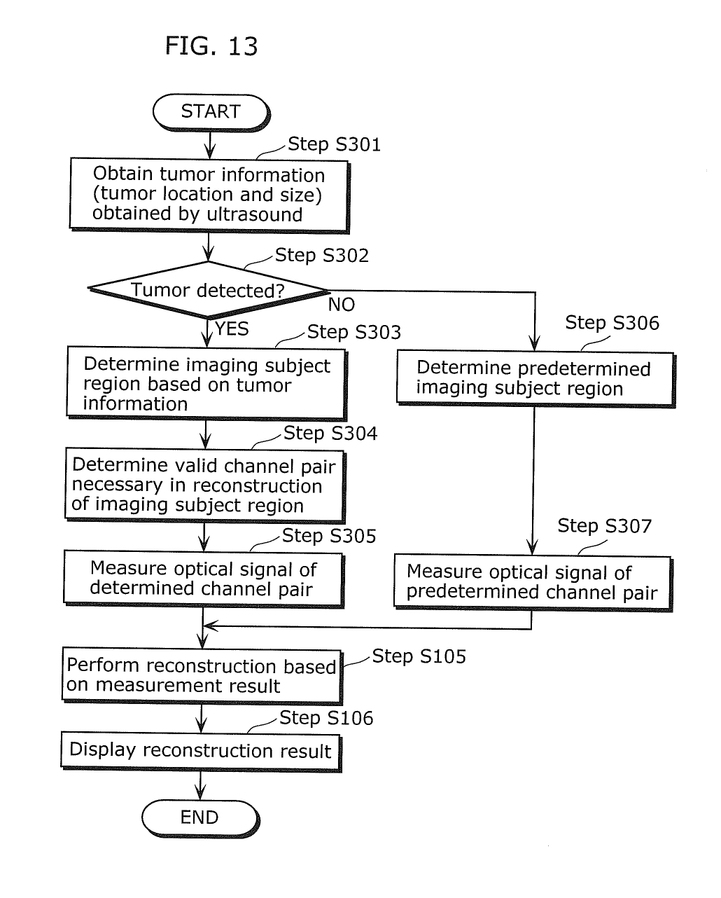

FIG. 13 is a flowchart showing an operation of the optical-combined imaging apparatus 100 in the present modification.

It should be noted that the optical-combined imaging apparatus 100 in the present modification is characterized in that the method of determining the imaging subject region is switched according to whether or not a tumor is detected through ultrasound.

First, the information obtainment unit 104 obtains the tumor information d1 obtained using ultrasound (step S301). This tumor information d1 includes information indicating whether or not a tumor was detected using ultrasound. The region determination unit 105 judges whether or not a tumor was detected using ultrasound, based on the above-described information included in the obtained tumor information d1 (step S302). Here, when the region determination unit 105 judges that a tumor was detected using ultrasound (YES in step S302), the region determination unit 105 automatically determines the imaging subject region based on the obtained tumor information d1 (step S303), and the channel pair determination unit 106 determines the valid channel pair required for the reconstruction of the imaging subject region (step S304), in the same manner as in steps S102 to S104 shown in FIG. 9 in the above-described embodiment. In addition, the optical signal measurement unit 107, using the valid channel pair determined for use in step S304, emits a laser beam (near infrared light) from the optical input channel, and measures the diffused light that has been propagated within the living body and has reached the optical output channel (step S305).

On the other hand, when it is judged in step S302 that a tumor was not detected (NO in step S302), the region determination unit 105 determines that a predetermined imaging subject region, that is, an imaging subject region determined in advance, is to be used (step S306). It should be noted that this predetermined imaging subject region is, for example, the entire imageable region. Next, the channel pair determination unit 106 performs the measurement of diffused light using the predetermined channel pairs corresponding to the predetermined imaging subject region determined in step S306 (step S307).

Subsequently, the image reconstruction unit 108 reconstructs the absorption coefficients within the determined imaging subject region based on the result of the measurement in step S305 or step S307, and images the reconstruction result (step S105). The display unit 103 displays the imaged reconstruction result (step S106).

In this manner, in the present modification, the imaging subject region is determined based on the presence or absence of a tumor, and thus imaging can be performed appropriately even when there seems to be no tumor.

(Modification 3)

Hereinafter, a third modification of the optical-combined imaging apparatus 100 in the present embodiment shall be described.

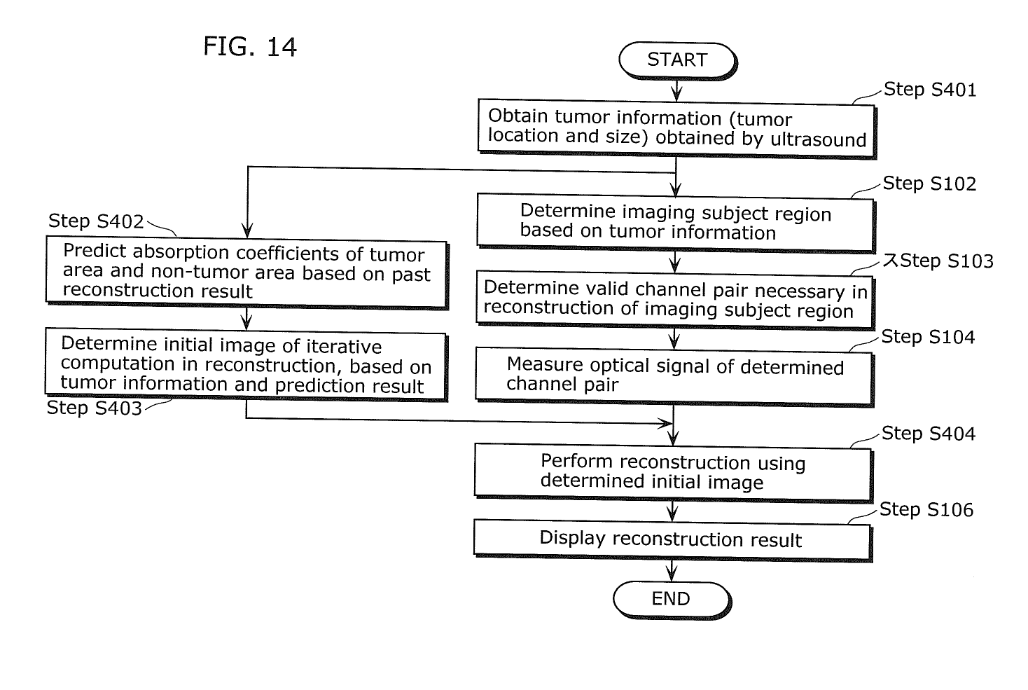

FIG. 14 is a flowchart showing an operation of the optical-combined imaging apparatus 100 in the present modification.

It should be noted that the optical-combined imaging apparatus 100 in the present modification is characterized in that the initial value for the absorption coefficient of each voxel in the reconstruction process is determined based on a past reconstruction result.

First, the information obtainment unit 104 obtains the tumor information d1 obtained using ultrasound (step S401). Next, the optical-combined imaging apparatus 100 executes the same processes as those in steps S102 to S104 shown in FIG. 9 in the above-described embodiment (step S102 to S104).

The image reconstruction unit 108 estimates the absorption coefficients for the tumor area and the non-tumor area based on a past reconstruction result, while the processes in steps S102 to S104 are being performed (step S402). A past measurement result during the same test or a measurement result during a past test can be used as a past reconstruction result. As a past measurement result during the same test, for example, when reconstruction is performed at plural locations while the user moves the combined probe 10c, a reconstruction result that is chronologically ahead during such movement can be used. Furthermore, as a measurement result during a past test, it is possible to use a preceding reconstruction result in tests performed plural times. It is to be noted that, since the absorption coefficient is different for a malignant tumor and a benign tumor, the absorption coefficient is set separately for both.

The image reconstruction unit 108 determines the initial value of each voxel in the reconstruction process, that is, the initial image of the iterative computation in the reconstruction, based on the absorption coefficients of the tumor area and the non-tumor area determined in step S402 and the location and size of the tumor indicated by the tumor information d1 obtained in step S401 (step S403).

Subsequently, the image reconstruction unit 108 performs reconstruction using the measurement result in step S104 and the initial values (initial image) determined in step S403, and reconstructs the absorption coefficients within the determined imaging subject region determined in step S102 and images the reconstruction result (step S404). The display unit 103 displays the imaged reconstruction result (step S106).

In this manner, in the present modification, the initial values for reconstruction are determined based on past reconstruction result, and since such past reconstruction result commonly approximates the reconstruction result obtainable in step S404, the processing load of the iterative computation in reconstruction can be reduced and the accuracy of reconstruction can be improved.

(Modification 4)

Hereinafter, a fourth modification of the optical-combined imaging apparatus 100 in the present embodiment shall be described.

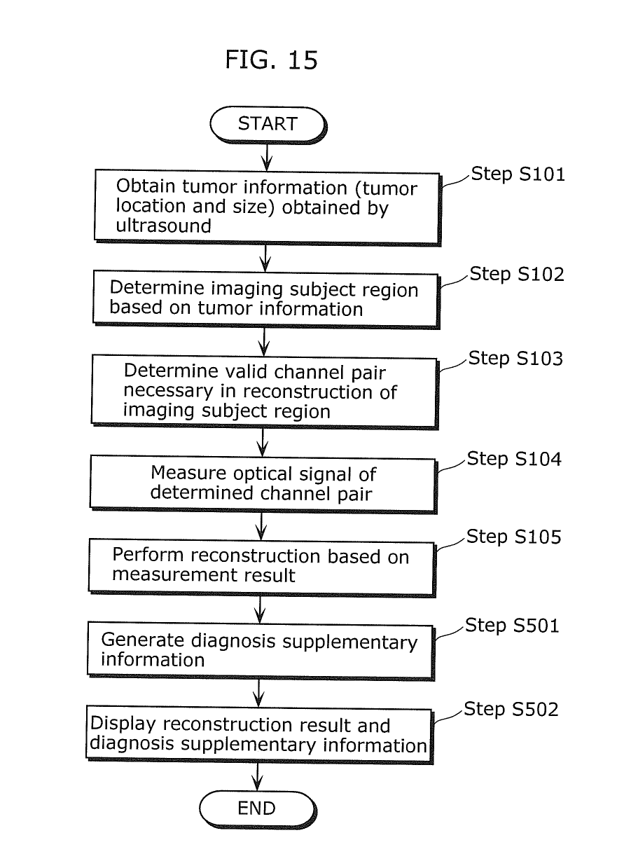

FIG. 15 is a flowchart showing an operation of the optical-combined imaging apparatus 100 in the present modification.

It should be noted that the optical-combined imaging apparatus 100 in the present modification is characterized in that, aside from the reconstruction result, diagnosis supplementary information based on such reconstruction result is displayed or presented.

First, in the same manner as the operation shown in FIG. 9 in the above-described embodiment, the optical-combined imaging apparatus 100 obtains the tumor information d1, determines the imaging subject region and the valid channel pair based on the tumor information d1, and performs reconstruction of the absorption coefficients in the imaging subject region based on the measurement result for that channel pair (steps S101 to S105).

Here, the image reconstruction unit 108 generates diagnosis supplementary information based on the reconstruction result in step S105, that is, the absorption coefficient of each voxel of the imaging subject region (step S501). For example, the image reconstruction unit 108 judges whether the tumor is malignant or benign based on the reconstruction result, and generates diagnosis supplementary information indicating the judgment result. Specifically, the image reconstruction unit 108 judges that the tumor is malignant when the absorption coefficients, which are the reconstruction result, are greater than a threshold, and judges that the tumor is benign when the absorption coefficients are equal to or less than the threshold. Alternatively, the image reconstruction unit 108 compares a past reconstruction result and a recent reconstruction result, and generates diagnosis supplementary information indicating a change in the tumor. For example, when the test subject is undergoing chemotherapy (radiation treatment, and the like), the image reconstruction unit 108 treats the aforementioned change in the tumor as an effect of the chemotherapy, and generates diagnosis supplementary information indicating such effect.

Subsequently, the display unit 103 displays the diagnosis supplementary information together with the imaged reconstruction result (step S502).

(Modification 5)

Hereinafter, a fifth modification of the optical-combined imaging apparatus 100 in the present embodiment shall be described.

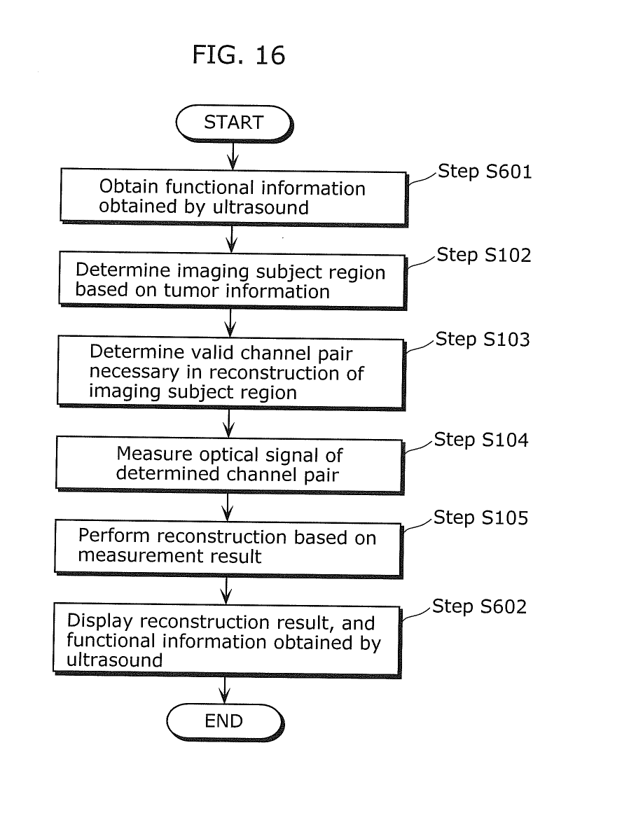

FIG. 16 is a flowchart showing an operation of the optical-combined imaging apparatus 100 in the present modification.

It should be noted that the optical-combined imaging apparatus 100 in the present modification is characterized in that the reconstruction result and functional information obtained by ultrasound (hereinafter called ultrasound functional information) are displayed or presented. Specifically, although ultrasound is used in the above-described embodiment for the purpose of obtaining structural information such as the location, size, and so on, of the tumor, that is, the tumor information d1, in the present modification, ultrasound is further used for the purpose of obtaining ultrasound functional information showing properties of tissues from temporal changes in ultrasound echo intensity. In addition, the present modification combines the use of the ultrasound functional information obtained by such ultrasound and the reconstruction result obtained using light (near infrared light). It is to be noted that the reconstruction result obtained by using light is functional information obtained using light and is called optical functional information.

First, the information obtainment unit 104 obtains the tumor information d1 obtained by ultrasound, from the ultrasound signal processing unit 102, and the image reconstruction unit 108 obtains ultrasound functional information from the ultrasound signal processing unit 102 (step S601). Specifically, the ultrasound signal processing unit 102 in the present modification generates, as the above-described tumor information d1, structural information which is information relating to structure, such as the location, shape, size, and so on, of the tumor, by analyzing the ultrasound signal measured by the ultrasound signal measurement unit 101, and generates ultrasound functional information indicating tissue properties (for example, blood flow volume, and so on), by analyzing the temporal change in the ultrasound signal. In addition, the ultrasound signal processing unit 102 outputs the tumor information d1 to the information obtainment unit 104, and outputs the tumor information d1 and the ultrasound functional information to the image reconstruction unit 108.

Next, the optical-combined imaging apparatus 100 executes the same processes as those in steps S102 to S105 shown in FIG. 9 in the above-described embodiment (step S102 to S105).

The display unit 103 displays, as optical functional information, the reconstruction result imaged in step S105, and displays the ultrasound functional information obtained in step S601 (step S602). Specifically, the display unit 103 obtains and displays functional information obtained using light and ultrasound, in addition to the image showing the reconstruction result obtained by ultrasound (image represented by the ultrasound image data d3). At this time, the image reconstruction unit 108 may define a new functional information parameter based on both the optical functional information and the ultrasound functional information, and cause the display unit 103 to display such functional information. Alternatively, when there is an association between both functional information, the image reconstruction unit 108 may check the conformity between the functional information, and judge that the reliability of the obtained functional information is high when the conformity is high. For example, this is possible when the ultrasound functional information reflects the blood flow volume in the tissue, in the same manner as the optical functional information. In this case, the image reconstruction unit 108 causes the display unit 103 to display the judgment result.

It should be noted that although the functional information obtained by ultrasound is displayed in the present modification, functional information obtained using a modal other than optical or ultrasound may be displayed. In addition, in the same manner as in Modification 4, diagnosis supplementary information may be generated based on obtained functional information and structural information, and such diagnosis supplementary information may also be displayed.

### Embodiment 2

The processes shown in the above-described Embodiment 1 and in the modifications thereof can be easily executed in an independent computer system by recording a program for realizing the optical-combined imaging method shown in the above-described Embodiment 1 and the modifications thereof on a recording medium such as a flexible disc, and so on.

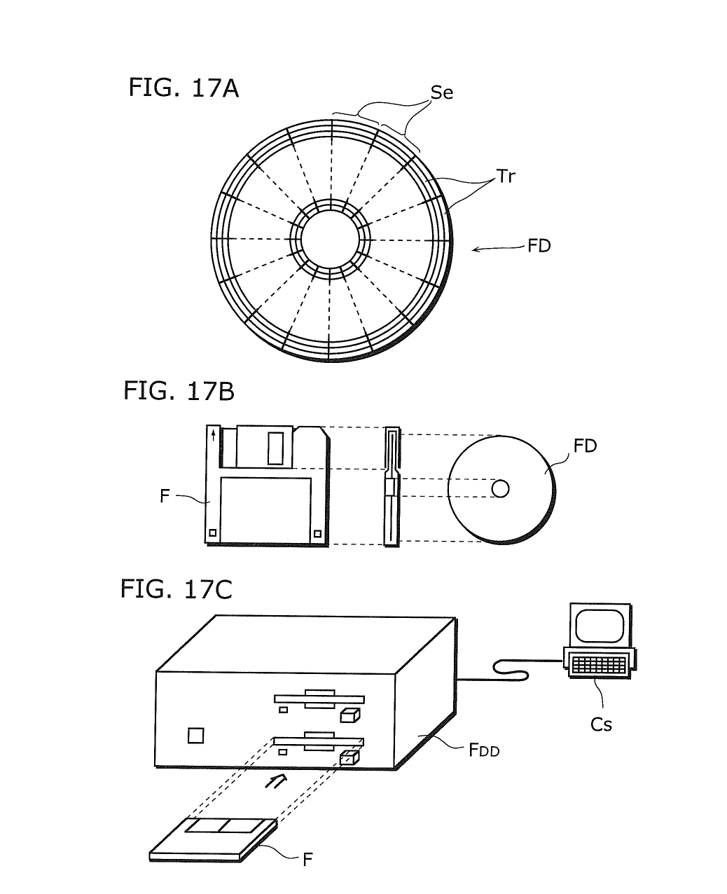

FIG. 17A to FIG. 17C are diagrams describing the case of executing the optical-combined imaging method in the above-described first embodiment and the modifications thereof through a computer system, by using a program recorded on a recording medium such as a flexible disc, and so on.

FIG. 17B shows the frontal external appearance and cross-sectional structure of a flexible disc F, and a flexible disc main body FD, and FIG. 17A shows an example of a physical format of the flexible disc main body FD which is a recording medium main body. The flexible disc main body FD is housed inside a case, and tracks TR are concentrically formed from the outer circumference towards the inner circumference. Each track is divided, in the angular direction, into 16 sectors Se. Therefore, in the flexible disc F which stores the aforementioned program, the aforementioned program is stored in the allotted region in the flexible disc main body FD.

Furthermore, FIG. 17C shows a configuration for performing the recording and reproduction of such program with respect to the flexible disc F. When the program which realizes the optical-combined method is to be recorded on the flexible disc F, the program is written from a computer system Cs via a flexible disk drive FDD. Furthermore, when constructing the optical-combined imaging method in the computer system Cs using the program inside the flexible disc F, the program is read from the flexible disc F and transferred to the computer system Cs using the flexible disk drive FDD.

It should be noted that although a flexible disc is used as the recording medium in the previous description, the same can be performed even when an optical disc is used. Furthermore, the recording medium is not limited to these, and the present invention can be implemented in the same manner as long as the recording medium is one that allows recording of the program, such as an IC card, a ROM cassette, and so on.

Although the optical-combined imaging method according to the present invention is described thus far based on the above-described Embodiment 1, Embodiment 2, and the modifications, the present invention is not limited to such embodiments and modifications. Variations of the above-described embodiments and modifications not exceeding the scope of the essence of the present invention that can be conceived by a person of ordinary skill in the art are included in the invention.

For example, although the optical-combined imaging apparatus 100 in Embodiment 1 includes constituent elements such as the information obtainment unit 104 as shown in FIG. 7, such constituent elements need not be included.

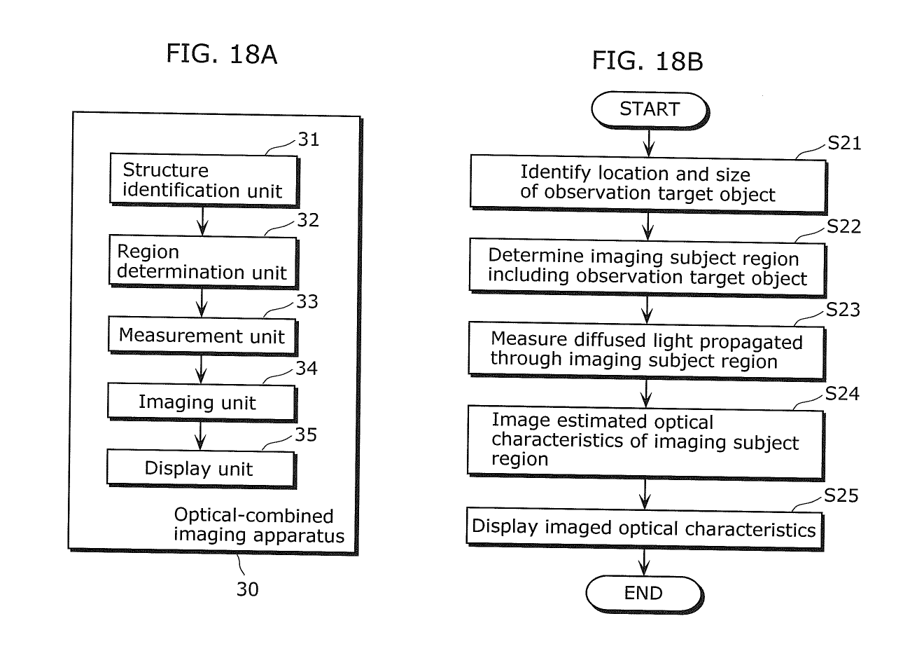

FIG. 18A is a block diagram showing a configuration of an optical-combined imaging apparatus according to the present invention.

An optical-combined imaging apparatus 30 is an optical-combined imaging apparatus which images the inside of a body tissue, using an optical-combined imaging method which is a combination of an imaging method and a structure identification method, the imaging method (i) measuring diffused light which is near infrared light emitted onto the body tissue and diffusing within the body tissue and (ii) imaging an optical characteristic within the body tissue, and the structure identification method identifying a structural characteristic within the body tissue by performing measurement that is different from the measurement of diffused light, the optical-combined imaging apparatus including: a structure identification unit 31 which identifies a location and a size of an observation target object that is present within the body tissue, using the structure identification method; a region determination unit 32 which determines an imaging subject region including the observation target object, based on the location and the size identified by the structure identification unit 31; a measurement unit 33 which measures the diffused light propagated through the imaging subject region determined by the region determination unit 32; an imaging unit 34 which estimates the optical characteristic within the imaging subject region based on a result of the measurement by the measurement unit 33, and images the estimated optical characteristic; and a display unit 35 which displays the optical characteristic imaged by the imaging unit 34.

For example, the optical-combined imaging apparatus 30 corresponds to the optical-combined imaging apparatus 100 in the above-described embodiments and modifications. Likewise, the structure identification unit 31 corresponds to the ultrasound signal measurement unit 101 and the ultrasound signal processing unit 102 in the above-described embodiments and modifications, and the region determination unit 32 corresponds to the region determination unit 105 in the above-described embodiments and modifications. The measurement unit 33 corresponds to the channel pair determination unit 106 and the optical signal measurement unit 107 in the above-described embodiments and modifications. Furthermore, the imaging unit 34 and the display unit 35 respectively correspond to the image reconstruction unit 108 and the display unit 103 in the above-described embodiments and modifications.

FIG. 18B is a flowchart showing operations in an optical-combined imaging method according to the present invention.

The optical-combined imaging method is an optical-combined imaging method which is a combination of an imaging method and a structure identification method, the imaging method (i) measuring diffused light which is near infrared light emitted onto body tissue and diffusing within the body tissue and (ii) imaging an optical characteristic within the body tissue, and the structure identification method identifying a structural characteristic within the body tissue by performing measurement that is different from the measurement of diffused light, the optical-combined imaging method including: an structure identification step S21 of identifying a location and a size of an observation target object that is present within the body tissue, using the structure identification method; a region determining step 522 of determining an imaging subject region including the observation target object, based on the location and the size identified in the structure identification step S21; a measurement step S23 of measuring the diffused light propagated through the imaging subject region determined in the region determining step S22; an imaging step S24 of estimating the optical characteristic within the imaging subject region based on a result of the measurement in the measurement step S23, and imaging the estimated optical characteristic; and a displaying step S25 of displaying the optical characteristic imaged in the imaging step S24.

In the optical-combined imaging apparatus 30 such as that described above, for example, an imaging subject region including an observation target object is determined based on the location and size of the observation target object identified by the structure identification method using ultrasound, and imaging using diffuse optical tomography is performed on the imaging subject region. Therefore, there is no need to set a wide fixed imaging subject region in advance so as to be able to cope with all locations and sizes of an observation target object as in the conventional techniques, and in the optical-combined imaging apparatus 30 according to the present invention, the imaging subject region can be set narrowly to an appropriate size in accordance with the observation target object. As a result, it is possible to prevent the estimation of an optical characteristic (for example, absorption coefficients), that is, performance of reconstruction of optical characteristics, based on measurement results of diffused light having a low SN ratio, and thus it is possible to improve the image quality of the image representing the imaged optical characteristic, that is, the reconstruction result, and shorten the time for imaging (particularly, the time for reconstruction).

Therefore, the present invention can produce the above-described function effect as in the optical-combined imaging apparatus 100, even without the information obtainment unit 104.

Furthermore, the optical-combined imaging apparatus 100 may further include a recording unit which records the reconstruction result in a memory. In addition to the reconstruction result, the recording unit may record, in the memory, the imaging subject region, information indicating the channel pair used in the measurement, the measurement result, or information indicating the positional relationship between the reconstruction result and the ultrasound image corresponding thereto.

Furthermore, although in Embodiment 1 the imaging subject region is determined, in step S102, from the tumor information d1 that is based on ultrasound structural information, the imaging subject region may be determined from tumor information d1 which is based on ultrasound functional information.

It should be noted that although the optical-combined imaging apparatus 100 in the above-described embodiments and modifications combines the use of ultrasound and light, an imaging method other than the imaging method using ultrasound may be combined with the imaging method using light (diffuse optical tomography), and another imaging method may be further combined with the imaging method which combines the use of light and ultrasound. As other imaging methods, there are, for example, mammography using X-ray or computed tomography (CT), magnetic resonance imaging (MRI), or positron emission tomography (PET), and so on.

It should be noted that although diffuse optical tomography is combined with the imaging method using ultrasound in the above-described embodiments and modifications, another optical imaging method different from diffuse optical tomography may be combined. As another optical imaging method, there is for example optical coherence tomography.

Furthermore, with the optical-combined imaging apparatus 100 in the above-described embodiments and modifications, observation is performed from outside the body. However, the optical-combined imaging apparatus 100 may be built into an endoscope.

Furthermore, applications of the optical-combined imaging apparatus 100 according to the present embodiment is not limited to the detection of tumors. For example, the optical-combined imaging apparatus 100 according to the present embodiment can also be applied to imaging of brain functions based on blood flow changes within the brain, the detection of disorders within the brain associated with bleeding, and so on. In addition, although, in the above-described embodiments and modifications, the optical-combined imaging apparatus 100 sets (determines) the imaging subject region based on the size, location, and so on, of the tumor, the imaging subject region may be set based on the size, position, and so on, of an observation target object that is different from a tumor.

Furthermore, each block (constituent elements) such as the region determination unit 105, the channel pair determination unit 106, and the image reconstruction unit 108 shown in FIG. 7 are typically implemented as a large scale integration (LSI) which is an integrated circuit. These functions blocks may be individually configured as single chips or may be configured so that a part or all of the function blocks are included in a single chip.

Although the name LSI is used here, there are instances where, due to the difference in degree of integration, the designations system LSI, super LSI, and ultra LSI are used.

Furthermore, the method of circuit integration is not limited to LSIs, and implementation through a dedicated circuit or a general-purpose processor is also possible. Field Programmable Gate Array (FPGA) that can be programmed after the LSI is manufactured, or a reconfigurable processor that allows reconfiguration of the connections and settings of the circuit cells within the LSI may be used.

In addition, depending on the emergence of circuit integration technology that replaces LSI due to progress in semiconductor technology or other derivative technology, it is obvious that such technology may be used to integrate the function blocks. Possibilities in this regard include the application of biotechnology and the like.

### INDUSTRIAL APPLICABILITY

The optical-combined imaging method and apparatus according to the present invention determine the imaging subject region for diffuse optical tomography based on the location, size, and so on, of a tumor obtained by an imaging method other than optical, and further determines the channel pair to be used in measuring diffused light, based on the imaging subject region, and thus enhance the image quality of the reconstruction result through the improvement of the SN ratio of the measured signal as well as shorten the time for imaging. Consequently, the optical-combined imaging method and apparatus according to the present invention shows high possibility for use particularly in the medical equipment industry.

### REFERENCE SIGNS LIST

10a Display device
10b Main device
10c Combined probe
30, 100 Optical-combined imaging apparatus
31 Structure identification unit
32, 105 Region determination unit
33 Measurement unit
34 Imaging unit
35, 103 Display unit
101 Ultrasound signal measurement unit
102 Ultrasound signal processing unit
104 Information obtainment unit
106 Channel pair determination unit
107 Optical signal measurement unit
108 Image reconstruction unit

1. An optical-combined imaging method which is a combination of an imaging method and a structure identification method, the imaging method (i) measuring diffused light which is near infrared light emitted onto body tissue and diffusing within the body tissue and (ii) imaging an optical characteristic within the body tissue, and the structure identification method identifying a structural characteristic within the body tissue by performing measurement that is different from the measurement of diffused light, said optical-combined imaging method comprising:
identifying a location and a size of an observation target object that is present within the body tissue, using the structure identification method;
determining an imaging subject region including the observation target object, based on the location and the size identified in said identifying;
measuring the diffused light propagated through the imaging subject region determined in said determining;
estimating the optical characteristic within the imaging subject region based on a result of the measurement in said measuring of the diffused light, and imaging the estimated optical characteristic; and
displaying the optical characteristic imaged in said imaging.

2. The optical-combined imaging method according to claim 1,
wherein said measuring of the diffused light includes:
emitting the near infrared light onto the body tissue so that the near infrared light is propagated through the imaging subject region determined in said determining; and
measuring, as the diffused light, the near infrared light emitted and propagated through the imaging subject region in said emitting.

3. The optical-combined imaging method according to claim 2, further comprising
selecting a channel pair that allows measurement of the diffused light propagated through the imaging subject region determined in said determining, from among channel pairs which are respective combinations of an emission channel for emitting near infrared light and a detection channel for detecting diffused light which is the near infrared light emitted from the corresponding emission channel and diffused within the body tissue, the channel pairs having mutually different regions through which the diffused light is propagated,
wherein in said emitting, the near infrared light is emitted from the emission channel of the channel pair selected in said selecting, and
in said measuring of the near infrared light, the diffused light propagated through the imaging subject region is measured by detecting the diffused light at the detection channel of the channel pair selected in said selecting.

4. The optical-combined imaging method according to claim 1,
wherein said measuring of the diffused light includes:
emitting the near infrared light onto the body tissue so that the near infrared light is propagated through a predetermined first region including the imaging subject region within the body tissue; and
measuring, as the diffused light, the near infrared light emitted and propagated through the predetermined first region in said emitting,
wherein in said estimating within the imaging subject region, the optical characteristic within the imaging subject region is estimated based only on a result of the measurement for the diffused light propagated through the imaging subject region, out of a result of the measurement in said measuring of the near infrared light.

5. The optical-combined imaging method according to claim 4, further comprising
selecting a channel pair that allows measurement of the diffused light propagated through the imaging subject region determined in said determining, from among channel pairs which are respective combinations of an emission channel for emitting near infrared light and a detection channel for detecting diffused light which is the near infrared light emitted from the corresponding emission channel and diffused within the body tissue, the channel pairs having mutually different regions through which the diffused light is propagated,
wherein in said emitting, the near infrared light is emitted from the respective emission channels of the channel pairs,
in said measuring of the near infrared light, the diffused light propagated through the predetermined first region is measured by detecting the diffused light at the respective detection channels of the channel pairs, and
in said estimating within the imaging subject region, the optical characteristic within the imaging subject region is estimated based only on a result of the measurement using the channel pair selected in said selecting, out of the result of the measurement in said measuring of the near infrared light.

6. The optical-combined imaging method according to claim 1,
wherein in said estimating within the imaging subject region, an absorption coefficient of the near infrared light for the body tissue is estimated as the optical characteristic, and the estimated optical characteristic is imaged.

7. The optical-combined imaging method according to claim 1,
wherein in said estimating within the imaging subject region, the imaging subject region is segmented into unit-regions, and the optical characteristic is estimated on a per unit-region basis.

8. The optical-combined imaging method according to claim 1, further comprising:
judging whether or not the observation target object is present within the body tissue, using the structure identification method;
measuring the diffused light propagated through a predetermined second region within the body tissue, when it is judged in said judging that the observation target object is not present;
estimating the optical characteristic within the predetermined second region based on a result of the measurement in said measuring of the diffused light propagated through the predetermined second region, and imaging the estimated optical characteristic; and
displaying the optical characteristic imaged in said estimating within the predetermined second region,
wherein in said identifying, the location and the size of the observation target object is identified when it is judged in said judging that the observation target object is present.

9. The optical-combined imaging method according to claim 1,
wherein the structure identification method is a method in which ultrasound emitted onto the body tissue and propagated within the body tissue is measured, and a structural characteristic within the body tissue is identified based on a result of the measurement.

10. The optical-combined imaging method according to claim 1, further comprising
identifying a biological characteristic of the observation target object based on the optical characteristic estimated in said estimating within the imaging subject region, and generating diagnosis supplementary information indicating the biological characteristic,
wherein in said displaying, the diagnosis supplementary information is further displayed.

11. The optical-combined imaging method according to claim 1, further comprising
identifying a functional characteristic within the body tissue by performing the measurement that is different from the measurement of diffused light, and generating functional information indicating the functional characteristic,
wherein in said displaying, the functional information is further displayed.

12. An optical-combined imaging apparatus which images the inside of a body tissue, using an optical-combined imaging method which is a combination of an imaging method and a structure identification method, the imaging method (i) measuring diffused light which is near infrared light emitted onto the body tissue and diffusing within the body tissue and (ii) imaging an optical characteristic within the body tissue, and the structure identification method identifying a structural characteristic within the body tissue by performing measurement that is different from the measurement of diffused light, said optical-combined imaging apparatus comprising:
a structure identification unit configured to identify a location and a size of an observation target object that is present within the body tissue, using the structure identification method;
a region determination unit configured to determine an imaging subject region including the observation target object, based on the location and the size identified by said structure identification unit;
a measurement unit configured to measure the diffused light propagated through the imaging subject region determined by said region determination unit;
an imaging unit configured to estimate the optical characteristic within the imaging subject region based on a result of the measurement by said measurement unit, and to image the estimated optical characteristic; and
a display unit configured to display the optical characteristic imaged by said imaging unit.

13. A non-transitory computer-readable recording medium on which program for an optical-combined imaging method is recorded, the optical-combined imaging method being a combination of an imaging method and a structure identification method, the imaging method (i) measuring diffused light which is near infrared light emitted onto body tissue and diffusing within the body tissue and (ii) imaging an optical characteristic within the body tissue, and the structure identification method identifying a structural characteristic within the body tissue by performing measurement that is different from the measurement of diffused light, the program causing a computer to execute:
identifying a location and a size of an observation target object that is present within the body tissue, using the structure identification method;
determining an imaging subject region including the observation target object, based on the location and the size identified in said identifying;
measuring the diffused light propagated through the imaging subject region determined in said determining;
estimating the optical characteristic within the imaging subject region based on a result of the measurement in said measuring of the diffused light, and imaging the estimated optical characteristic; and
displaying the optical characteristic imaged in said imaging.

14. An integrated circuit which images the inside of a body tissue, using an optical-combined imaging method which is a combination of an imaging method and a structure identification method, the imaging method (i) measuring diffused light which is near infrared light emitted onto the body tissue and diffusing within the body tissue and (ii) imaging an optical characteristic within the body tissue, and the structure identification method identifying a structural characteristic within the body tissue by performing measurement that is different from the measurement of diffused light, said integrated circuit comprising:
a structure identification unit configured to identify a location and a size of an observation target object that is present within the body tissue, using the structure identification method;
a region determination unit configured to determine an imaging subject region including the observation target object, based on the location and the size identified by said structure identification unit;
a measurement unit configured to measure the diffused light propagated through the imaging subject region determined by said region determination unit;
an imaging unit configured to estimate the optical characteristic within the imaging subject region based on a result of the measurement by said measurement unit, and to image the estimated optical characteristic; and
a display unit configured to display the optical characteristic imaged by said imaging unit.
